SEQUENCE LISTING (63 sequences)

SEQ ID NO: 1 - RNA, 20 nt, synthetic construct
  source 1..20: mol_type = other RNA
  ncRNA 1..20: ncRNA_class = sgRNA
gcagctctcgccgcaactcc

SEQ ID NO: 2 - DNA, 142 bp, synthetic construct
  source 1..142: mol_type = other DNA
ggctgccggcgggagcagttccgggtgcggtgcgcgccgggggcgggcgagggggcggtgtcctggccatggccggcctgtcggacctggaattgcggcgagagcggcaggccctgggcttccagccaggccccatcaccga

SEQ ID NO: 3 - RNA, 20 nt, synthetic construct
  source 1..20: mol_type = other RNA
  ncRNA 1..20: ncRNA_class = sgRNA
ctcgacgcccatccggagac

SEQ ID NO: 4 - RNA, 20 nt, synthetic construct
  source 1..20: mol_type = other RNA
  ncRNA 1..20: ncRNA_class = sgRNA
ccttcggggaatgcctgccg

SEQ ID NO: 5 - DNA, 167 bp, synthetic construct
  source 1..167: mol_type = other DNA
ctaacgcagcgttagcacgtggtaaacgttcaatggaatgttgatttacttaatggatgagctcaatggtgtaagaaaaccaccgccattagctagcataacttcgtataatgtatgctatacgaagttatatagagatccggcccacttgccagtctccggatggg

SEQ ID NO: 6 - DNA, 167 bp, synthetic construct
  source 1..167: mol_type = other DNA
cccacacctgagccacaggcagggtcagactccttttgacaaatgaaggcccggtggtgccagttttgctgctacaaagctgagaccacggactagtataacttcgtataatgtatgctatacgaagttatcaggcattccccgaaggcagggaagacaagaggggc

SEQ ID NO: 7 - DNA, 21 bp, synthetic construct
  source 1..21: mol_type = other DNA
tgttgttacgcccagagtctt

SEQ ID NO: 8 - DNA, 20 bp, synthetic construct
  source 1..20: mol_type = other DNA
ctatgtccgccatgatgaaa

SEQ ID NO: 9 - DNA, 20 bp, synthetic construct
  source 1..20: mol_type = other DNA
aaagccacacgcacactctt

SEQ ID NO: 10 - DNA, 20 bp, synthetic construct
  source 1..20: mol_type = other DNA
gtgccagttttgctgctaca

SEQ ID NO: 11 - DNA, 20 bp, synthetic construct
  source 1..20: mol_type = other DNA
aagactggagcaaaatgatg

SEQ ID NO: 12 - DNA, 20 bp, synthetic construct
  source 1..20: mol_type = other DNA
gtcaggttagcaaaatcctc

SEQ ID NO: 13 - DNA, 19 bp, synthetic construct
  source 1..19: mol_type = other DNA
atgactgagtacctgaacc

SEQ ID NO: 14 - DNA, 21 bp, synthetic construct
  source 1..21: mol_type = other DNA
atatagttccacaaaggcatc

SEQ ID NO: 15 - DNA, 20 bp, synthetic construct
  source 1..20: mol_type = other DNA
acgagttcaacgaaacttac

SEQ ID NO: 16 - DNA, 20 bp, synthetic construct
  source 1..20: mol_type = other DNA
tagatcctgtcaatgccttc

SEQ ID NO: 17 - DNA, 21 bp, synthetic construct
  source 1..21: mol_type = other DNA
ctgaccgatcattacaaacac

SEQ ID NO: 18 - DNA, 21 bp, synthetic construct
  source 1..21: mol_type = other DNA
agacacttcatagggatcaac

SEQ ID NO: 19 - DNA, 18 bp, synthetic construct
  source 1..18: mol_type = other DNA
ctgcagatccatttcacg

SEQ ID NO: 20 - DNA, 21 bp, synthetic construct
  source 1..21: mol_type = other DNA
ttaggattcgaaatgaggatg

SEQ ID NO: 21 - DNA, 21 bp, synthetic construct
  source 1..21: mol_type = other DNA
catgaagaactttcagaaggg

SEQ ID NO: 22 - DNA, 21 bp, synthetic construct
  source 1..21: mol_type = other DNA
tagatatggtacaaggaagcc

SEQ ID NO: 23 - DNA, 21 bp, synthetic construct
  source 1..21: mol_type = other DNA
cttctgtcatcaaagaggttg

SEQ ID NO: 24 - DNA, 21 bp, synthetic construct
  source 1..21: mol_type = other DNA
caaaggtgaagtcttgttctc

SEQ ID NO: 25 - DNA, 20 bp, synthetic construct
  source 1..20: mol_type = other DNA
ggtactctcatctcgacttc

SEQ ID NO: 26 - DNA, 20 bp, synthetic construct
  source 1..20: mol_type = other DNA
gccagatttttagcttccag

SEQ ID NO: 27 - DNA, 20 bp, synthetic construct
  source 1..20: mol_type = other DNA
acctgatgatacccaactac

SEQ ID NO: 28 - DNA, 20 bp, synthetic construct
  source 1..20: mol_type = other DNA
ctgtggcaccttttattctg

SEQ ID NO: 29 - DNA, 20 bp, synthetic construct
  source 1..20: mol_type = other DNA
ttttgtctatttggggacag

SEQ ID NO: 30 - DNA, 20 bp, synthetic construct
  source 1..20: mol_type = other DNA
catagttcctgttggtgaag

SEQ ID NO: 31 - DNA, 20 bp, synthetic construct
  source 1..20: mol_type = other DNA
tgcaatcttctttttacccc

SEQ ID NO: 32 - DNA, 20 bp, synthetic construct
  source 1..20: mol_type = other DNA
caagagcaaagtacaagtcc

SEQ ID NO: 33 - DNA, 21 bp, synthetic construct
  source 1..21: mol_type = other DNA
gactgtgagagaamacagatg

SEQ ID NO: 34 - DNA, 21 bp, synthetic construct
  source 1..21: mol_type = other DNA
cacattagctatgtactcctg

SEQ ID NO: 35 - DNA, 21 bp, synthetic construct
  source 1..21: mol_type = other DNA
aggtcggtgtgaacggatttg

SEQ ID NO: 36 - DNA, 23 bp, synthetic construct
  source 1..23: mol_type = other DNA
tgtagaccatgtagttgaggtca

SEQ ID NO: 37 - DNA, 20 bp, synthetic construct
  source 1..20: mol_type = other DNA
ccatccaatcggtagtagcg

SEQ ID NO: 38 - DNA, 20 bp, synthetic construct
  source 1..20: mol_type = other DNA
gtaacccgttgaaccccatt

SEQ ID NO: 39 - protein, 41 aa, Homo sapiens
RMRKFKNKGKDTAELRRRRVEVSVELRKAKKDEQILKRRNV

SEQ ID NO: 40 - protein, 8 aa, Homo sapiens
VSRKRPRP

SEQ ID NO: 41 - protein, 8 aa, Homo sapiens
PQPKKKPL

SEQ ID NO: 42 - protein, 12 aa, Mus sp.
SALIKKKKKMAP

SEQ ID NO: 43 - protein, 7 aa, Alphainfluenzavirus influenzae
PKQKKRK

SEQ ID NO: 44 - protein, 10 aa, Hepatitis B virus
RKLKKKIKKL

SEQ ID NO: 45 - protein, 10 aa, Mus sp.
REKKKFLKRR

SEQ ID NO: 46 - protein, 20 aa, Homo sapiens
KRKGDEVDGVDEVAKKKSKK

SEQ ID NO: 47 - protein, 17 aa, Homo sapiens
RKCLQAGMNLEARKTKK

SEQ ID NO: 48 - DNA, 60 bp, synthetic construct
  source 1..60: mol_type = other DNA
tagcaataaaggatcgtttattttcattggaagcgtgtgttggttttttgatcaggcgcg

SEQ ID NO: 49 - DNA, 48 bp, synthetic construct
  source 1..48: mol_type = other DNA
aagcgtcctgctgctactaagaaagctggtcaagctaagaaaaagaaa

SEQ ID NO: 50 - DNA, 51 bp, synthetic construct
  source 1..51: mol_type = other DNA
atggccccaaagaagaagcggaaggtcggtatccacggagtcccagcagcc

SEQ ID NO: 51 - DNA, 1155 bp, synthetic construct
  source 1..1155: mol_type = other DNA
catgcaagctgtagccaaccactagaactatagctagagtcctgggcgaacaaacgatgctcgccttccagaaaaccgaggatgcgaaccacttcatccggggtcagcaccaccggcaagcgccgcgacggccgaggtcttccgatctcctgaagccagggcagatccgtgcacagcaccttgccgtagaagaacagcaaggccgccaatgcctgacgatgcgtggagaccgaaaccttgcgctcgttcgccagccaggacagaaatgcctcgacttcgctgctgcccaaggttgccgggtgacgcacaccgtggaaacggatgaaggcacgaacccagttgacataagcctgttcggttcgtaaactgtaatgcaagtagcgtatgcgctcacgcaactggtccagaaccttgaccgaacgcagcggtggtaacggcgcagtggcggttttcatggcttgttatgactgtttttttgtacagtctatgcctcgggcatccaagcagcaagcgcgttacgccgtgggtcgatgtttgatgttatggagcagcaacgatgttacgcagcagcaacgatgttacgcagcagggcagtcgccctaaaacaaagttaggtggctcaagtatgggcatcattcgcacatgtaggctcggccctgaccaagtcaaatccatgcgggctgctcttgatcttttcggtcgtgagttcggagacgtagccacctactcccaacatcagccggactccgattacctcgggaacttgctccgtagtaagacattcatcgcgcttgctgccttcgaccaagaagcggttgttggcgctctcgcggcttacgttctgcccaagtttgagcagccgcgtagtgagatctatatctatgatctcgcagtctccggcgagcaccggaggcagggcattgccaccgcgctcatcaatctcctcaagcatgaggccaacgcgcttggtgcttatgtgatctacgtgcaagcagattacggtgacgatcccgcagtggctctctatacaaagttgggcatacgggaagaagtgatgcactttgatatcgacccaagtaccgccacctaacaattcgttcaagccgagatcggcttcccggccgcggagttgttcggtaaattgtcacaacgccg

SEQ ID NO: 52 - RNA, 1512 nt, Homo sapiens
  source 1..1512: mol_type = mRNA
atggccggcctgtcggacctggaactgcggcgggagctgcaggccctgggcttccagccaggacccatcaccgacaccacccgggatgtctaccgcaacaagctgcgccgcctgcggggcgaggcccggctgcgcgacgaggagcggctgcgggaggaggcccggccgcggggcgaggagcggttacgggaagaggcccggttacgcgaggatgcgccgctgcgcgcccggcccgccgcggcctctccgcgggcggagccctggctctcccagccggcctcgggctcggcctacgcgacccctggggcctacggtgatatccggccctccgcggcttcctgggtagggagccgcggcctcgcctatcctgcccgcccggcgcaactcaggcgccgcgcctcggtccggggcagctccgaggaggacgaggacgcccggacgcccgacagggccacgcagggcccgggtctcgcggcccgccgctggtgggcagcgtctcccgccccggcgcggctgccttcctccctcctcggtcccgacccgcgcccgggcctgcgggcgactcgagcgggccctgctggcgcggcgagggcccggcctgaggtggggcgccggctggagcgctggctctctcggcttctgctctgggccagcctagggctactgctcgtcttcctgggcatcctttgggtgaagatgggcaagccctcagcgccgcaggaggcggaggacaacatgaagttattgccagtggactgtgagagaaaaacagatgagttctgtcaggccaagcagaaggcagccttgctggagctgctgcatgaactctacaatttcctggccatccaagctggtaattttgagtgtggaaatccagagaatctaaaaagcaaatgcattcctgttatggaagcccaagaatatatagccaatgtgaccagcagctcctccgccaagtttgaagccgcactgacctggatactgagcagtaacaaggacgtgggcatctggttgaaaggagaagaccagtctgaattggtgacgactgtggacaaggtggtctgcctggaatctgcccacccccgcatgggtgttggctgccgcctgagccgggccttgctcactgctgtcaccaacgtgctcatcttcttctggtgcttggcttttttgtgggggctcctaattctcctaaaatatcggtggcgaaagttagaagaggaggaacaagccatgtatgagatggtgaagaagattatagacgtggtccaggaccattacgtggactgggagcaggacatggagcgctatccatatgtaggcatcctgcacgtgcgcgacagcttgatccctccacagagccggaggcgcatgaagcgtgtctgggaccgagctgtggagttcctggcctccaacgaatcccggatccagacggagtcccaccgcgttgcaggagaggacatgctggtgtggagatggactaagccctcttccttctctgactcagagcgataa

SEQ ID NO: 53 - protein, 503 aa, Homo sapiens
MAGLSDLELRRELQALGFQPGPITDTTRDVYRNKLRRLRGEARLRDEERLREEARPRGEERLREEARLREDAPLRARPAAASPRAEPWLSQPASGSAYATPGAYGDIRPSAASWVGSRGLAYPARPAQLRRRASVRGSSEEDEDARTPDRATQGPGLAARRWWAASPAPARLPSSLLGPDPRPGLRATRAGPAGAARARPEVGRRLERWLSRLLLWASLGLLLVFLGILWVKMGKPSAPQEAEDNMKLLPVDCERKTDEFCQAKQKAALLELLHELYNFLAIQAGNFECGNPENLKSKCIPVMEAQEYIANVTSSSSAKFEAALTWILSSNKDVGIWLKGEDQSELVTTVDKVVCLESAHPRMGVGCRLSRALLTAVTNVLIFFWCLAFLWGLLILLKYRWRKLEEEEQAMYEMVKKIIDVVQDHYVDWEQDMERYPYVGILHVRDSLIPPQSRRRMKRVWDRAVEFLASNESRIQTESHRVAGEDMLVWRWTKPSSFSDSER

SEQ ID NO: 54 - RNA, 1536 nt, Mus sp.
  source 1..1536: mol_type = mRNA
atggccggcctgtcggacctggagttgcggcgagagctgcaggccctgggcttccagccaggccccatcaccgacaccacgcggaacgtctaccgcaacaagctgcgccgcctgcggggcgaggcccggctgcgcgacgacgagcggctgcgggaggacgccgggccgcgggaggacgccgggccgcggggccccgagcggcagcgggaggaggcccggctacgcgaggaagcgccgctgcgcgcgcggcccgccgccagcgtcctgcgctcggagccctggccgctgtcgccttccccgccggcgcctagcgcggcctccgacgcctcggggccgtacggcaacttcggggcctccgcctctccctgggccgcgagccgcggcctctcctacccgccccacgccgggcccgggccgctgcgacgccgcgcctcggtccggggcagctcggaggatgacgaggatacacgaacgccggacaggcacgccccgggccggggccgccactggtgggccccgccgtcggcctctgcgcggccgcactcggcgctcctcggcgccgacgctcgccccggcctgaagggctcgcgcaccggctcggcgggcgccgggcggacccgacccgaggtgggccggtggctggagcgctgcctgtctcggctcctgctctgggccagcctggggctgctgctcggcttcttggccatcctgtgggtgaagatgggcaagccctcggcgccgcaggaggcggaggacaacatgaaattgttgccggtcgactgtgagagaaaaacagatgagttctgccaggccaagcagaaggcggccctgttggagctgctgcacgaactgtacaacttcctggccatccaggcaggtaattttgaatgtggcaacccggagaagctgaagagcaagtgcatcccagtcctggaggcgcaggagtacatagctaatgtgaccagcagtccctcttcgaggtttaaggctgcgctgacctggatcctgagcagcaacaaggacgtgggcatctggctgaaaggggaagacccatctgagctggcgactacagtggacaaagtggtctgcctggagtccgcccggccccggatgggcataggctgccgcctcagccgcgccctgctcaccgctgtcacccacgtgctcatcttcttctggtgcctggccttcctgtgggggctgctgatcctcctgaagtaccgctggcggaagctggaggaggaggagcaggccatgtatgagatggtgaagaagatcatagatgtggttcaggaccactatgtggactgggaacaggacatggagcgctacccctacgtgggcatcctccacgtgcgggacagtctaatccccccacagagccggcggcgcatgaagcgagtatgggaccgtgctgtggaattcctggcttccaatgaatctcggatccagactgagtcccaccgagtggccggggaggacatgctggtgtggagatggactaagccttcctctttctctgattcagagcgatag

SEQ ID NO: 55 - protein, 511 aa, Mus sp.
MAGLSDLELRRELQALGFQPGPITDTTRNVYRNKLRRLRGEARLRDDERLREDAGPREDAGPRGPERQREEARLREEAPLRARPAASVLRSEPWPLSPSPPAPSAASDASGPYGNFGASASPWAASRGLSYPPHAGPGPLRRRASVRGSSEDDEDTRTPDRHAPGRGRHWWAPPSASARPHSALLGADARPGLKGSRTGSAGAGRTRPEVGRWLERCLSRLLLWASLGLLLGFLAILWVKMGKPSAPQEAEDNMKLLPVDCERKTDEFCQAKQKAALLELLHELYNFLAIQAGNFECGNPEKLKSKCIPVLEAQEYIANVTSSPSSRFKAALTWILSSNKDVGIWLKGEDPSELATTVDKVVCLESARPRMGIGCRLSRALLTAVTHVLIFFWCLAFLWGLLILLKYRWRKLEEEEQAMYEMVKKIIDVVQDHYVDWEQDMERYPYVGILHVRDSLIPPQSRRRMKRVWDRAVEFLASNESRIQTESHRVAGEDMLVWRWTKPSSFSDSER

SEQ ID NO: 56 - protein, 8 aa, Homo sapiens
PPKKARED

SEQ ID NO: 57 - protein, 5 aa, Alphainfluenzavirus influenzae
DRLRR

SEQ ID NO: 58 - RNA, 33 nt, synthetic construct
  source 1..33: mol_type = other RNA
  ncRNA 1..33: ncRNA_class = sgRNA
tcggacctggagttgcggcgagagctgcaggcc

SEQ ID NO: 59 - RNA, 33 nt, synthetic construct
  source 1..33: mol_type = other RNA
  ncRNA 1..33: ncRNA_class = sgRNA
tcggacctggaattgcggcgagagcggcaggcc

SEQ ID NO: 60 - protein, 11 aa, synthetic construct
SDLELRRELQA

SEQ ID NO: 61 - protein, 11 aa, synthetic construct
SDLELRRERQA

SEQ ID NO: 62 - protein, 9 aa, Mus sp.
LELRRERQA

SEQ ID NO: 63 - DNA, 28 bp, Mus sp.
  source 1..28: mol_type = other DNA
cctggaattgcggcgagagcggcaggcc

(19) United States
(12) Patent Application Publication
(10) Pub. No.: US 2026/0174900 A1
(43) Pub. Date: Jun. 25, 2026

(54) GENE THERAPY FOR LEMD2 CARDIOMYOPATHY

(71) Applicant: The Board of Regents of The University of Texas System, Austin, TX (US)

(72) Inventors: Eric N. OLSON, Dallas, TX (US); Xurde Menendez CARAVIA, Dallas, TX (US)

(73) Assignee: The Board of Regents of The University of Texas System, Austin, TX (US)

(21) Appl. No.: 19/125,436

(22) PCT Filed: Oct. 27, 2023
(86) PCT No.: PCT/US2023/078001

§ 371 Date: Apr. 29, 2025

Related U.S. Application Data
(60) Provisional application No. 63/381,837, filed on Nov. 1, 2022.

(51) Int. Cl. A61K 48/00; A01K 67/0278; A61K 9/00; A61K 38/00; A61P 7/00; C07K 14/705; C12N 9/22; C12N 15/11; C12N 15/86

(52) U.S. Cl. CPC A61K 48/0058; A01K 67/0278; A61K 9/0019; A61K 48/0075; A61P 7/00; C07K 14/705; C12N 9/226; C12N 15/111; C12N 15/86; A01K 2217/072; A01K 2227/105; A01K 2267/0306; A61K 38/00; C12N 2310/20; C12N 2750/14143; C12N 2800/22

(57) ABSTRACT

Provided herein are composition and methods for gene therapy of LEM domain-containing protein 2 (LEMD2) cardiomyopathy. Viral vectors are used to deliver LEMD2 reading frame for gene augmentation in cardiac cells that harbor loss-of-function LEMD2 mutations. A murine model for LEMD2 cardiomyopathy is also described.

### PRIORITY CLAIM

This application claims benefit of priority to U.S. Ser. No. 63/381,837, filed Nov. 1, 2022, the entire contents of which are hereby incorporated by reference.

### REFERENCE TO A SEQUENCE LISTING

This application contains a Sequence Listing XML, which has been submitted electronically and is hereby incorporated by reference in its entirety. Said XML Sequence Listing, created on Oct. 26, 2023, is named UTFDP4182WO_ST26.xml and is ˜63 kilobytes in size.

### BACKGROUND

### 1. Field

The present invention relates generally to the fields of molecular biology, medicine, and genetics. More particularly, it concerns compositions and methods for gene therapy of LEMD2-associated cardiomyopathy.

### 2. Description of Related Art

The nuclear envelope (NE) constitutes the boundary between the nucleus and the cytoplasm in eukaryotic cells. The inner (INM) and outer (ONM) nuclear membranes contain nuclear envelope proteins (NEPs) connected to the underlying nuclear lamina, a protein meshwork composed by lamin filaments that provide physical support for the entire structure (Ungricht & Kutay, 2017). Collectively, NEPs execute a wide variety of essential cellular functions, such as mechanotransduction and chromatin organization (Pawar & Kutay, 2021). At least 80 NEPs have been identified in the rodent liver (Schirmer et al., 2003) and several hundred are present in muscle cells (Wilkie et al., 2011; Cheng et al., 2019). The plethora of NEPs in muscle reflects their functional relevance in this tissue. To date, hundreds of mutations in lamins and NEPs have been shown to cause human pathological syndromes (Janin et al., 2017). Paradoxically, although lamins and several NEPs are ubiquitously expressed, their genetic mutations cause cardiac- or skeletal muscle-specific phenotypes, in most cases (Janin & Gache, 2018). For example, mutations in the gene encoding the ubiquitously expressed NEP emerin, cause a severe disease named Emery-Dreifuss Muscular Dystrophy (EDMD), which is characterized by skeletal muscle wasting and cardiac pathology (Bione et al., 1994; Brull et al., 2018; Shin & Worman, 2021). In this regard, various hypotheses have been proposed to explain the etiology of these pathologies collectively known as envelopathies (Gerbino et al., 2018). The “mechanical stress” hypothesis proposes that mutations in NEPs decrease the rigidity of the NE, affecting mechanotransduction and sensitizing cells to mechanical stress. This may explain why cardiac and skeletal muscles are particularly sensitive to alterations in NEPs. On the other hand, the “gene expression” hypothesis suggests that NEP mutations induce alterations in important signaling pathways and chromatin organization, which lead to aberrant gene expression patterns, causing pathological phenotypes. The lack of a complete understanding of the molecular basis of NE myopathies poses challenges to development of mutation-specific therapies for affected patients.

The lamina-associated polypeptide-emerin-MAN1 (LEM) family constitutes a group of proteins located in the INM that share the evolutionarily-conserved LEM structural domain (Braun & Barrales, 2016). LEM domain containing protein 2 (LEMD2), which is expressed ubiquitously, is characterized by the presence of the LEM domain and two transmembrane domains. A series of in vitro studies revealed its ability to associate with DNA-binding proteins such as lamins and barrier-to-autointegration factor (BAF), which implicates LEMD2 as a mediator of the interaction between chromatin and the NE (Brachner et al., 2005; Ulbert et al., 2006; Huber et al., 2009). Recent reports highlighted the requirement for LEMD2 in NE reformation after cell division, as well as nuclear integrity and chromatin stabilization (von Appen et al., 2020; Vietri et al., 2020). Notably, the first study in rodents regarding Lemd2 showed that mice lacking this gene die during embryogenesis due to abnormal heart development (Tapia et al., 2015). In humans, LEMD2 mutations have also been linked to cardiac disease. Specifically, a LEMD2 c.T38>G homozygous missense mutation is associated with arrhythmic cardiomyopathy, cataracts, and sudden death (Boone et al., 2016; Abdelfatah et al., 2019). This nucleotide transversion (T38>G) results in the exchange of a leucine for an arginine in the LEM domain, which is essential for LEMD2 function (von Appen et al., 2020). Together, these findings highlight the importance of LEMD2 for cardiac function even though its specific roles in the heart remain unknown.

### SUMMARY

In accordance with the present disclosure, in certain embodiments, there is provided an expression construct comprising a coding region for LEM domain-containing protein 2 (LEMD2) under the control of a heterologous promoter.

In some aspects, said expression construct is a non-viral expression construct. In certain aspects, said expression construct is a viral expression construct. In some aspects, said heterologous protein is a constitutive promoter or an inducible promoter. In certain aspects, the promoter is a muscle-specific promoter. In particular aspects, the muscle-specific promoter is a cardiac troponin T (cTnT) promoter. In some aspects, said viral expression construct is a retroviral construct, an adenoviral construct, an adeno-associated viral construct, a poxviral construct, or a herpesviral construct. In specific aspects, the adeno-associated viral construct comprises a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11), MyoAAV, or any combination thereof. In some aspects, the AAV vector comprises a sequence isolated or derived from an AAV vector of serotype 9 (AAV9). In certain aspects, the AAV vector comprises a sequence isolated or derived from an AAV vector of serotype 2 (AAV2). In some aspects, the AAV vector comprises a sequence isolated or derived from an AAV2 and a sequence isolated or derived from an AAV9. In some aspects, the viral vector is optimized for expression in mammalian cells. In certain aspects, the vector is optimized for expression in human cells.

A further embodiment provides a composition comprising the expression construct of the present embodiments and aspects thereof (e.g., an expression construct comprising a coding region for LEM domain-containing protein 2 (LEMD2) under the control of a heterologous promoter).

Another embodiment provides a cell comprising the expression construct of the present embodiments and aspects thereof (e.g., an expression construct comprising a coding region for LEM domain-containing protein 2 (LEMD2) under the control of a heterologous promoter). In some aspects, the cell is a human cell. In certain aspects, the cell is a mouse cell. In some aspects, the human cell is a cardiomyocyte. In particular aspects, the cell or human cell is an induced pluripotent stem (iPS) cell.

A further embodiment provides a composition comprising the cell of the present embodiments and aspects thereof (e.g., a cell comprising an expression construct comprising a coding region for LEM domain-containing protein 2 (LEMD2) under the control of a heterologous promoter).

In yet another embodiment, there is provided a method of expressing LEM domain-containing protein 2 (LEMD2) in a cell comprising delivering an expression construct of the present embodiments or aspects thereof (e.g., an expression construct comprising a coding region for LEM domain-containing protein 2 (LEMD2) under the control of a heterologous promoter) to said cell.

Another embodiment provides a method of treating or preventing LEM domain-containing protein 2 (LEMD2) cardiomyopathy in a subject comprising administering to the subject a therapeutically effective amount of a composition comprising the expression construct of the present embodiments or aspects thereof (e.g., an expression construct comprising a coding region for LEM domain-containing protein 2 (LEMD2) under the control of a heterologous promoter).

In some aspects, the composition is administered locally. In certain aspects, the composition is administered directly to cardiac tissue. In certain aspects, the composition is administered by an infusion or injection. In some aspects, the composition is administered systemically. In some aspects, the composition is administered by an intravenous infusion or injection. In certain aspects, administration of said expression construct results one or more of an increase in systolic LVAW thickness as compared with Lemd2 KI/KI untreated mice, a smaller LVID as compared with Lemd2 KI/KI untreated mice, improvement in cardiac functional parameters (e.g., EF, FS and LV volume) as compared with untreated Lemd2 KI/KI animals and/or amelioration of DCM phenotype and fibrotic accumulation compared with Lemd2 KI/KI untreated mice. In some aspects, the subject is a neonate, an infant, a child, a young adult, or an adult. In certain aspects, the subject is male. In some aspects, the subject is female.

A further embodiment provides the use of a therapeutically effective amount of a composition comprising an expression construct of the present embodiments or aspects thereof (e.g., an expression construct comprising a coding region for LEM domain-containing protein 2 (LEMD2) under the control of a heterologous promoter) for treating or preventing LEM domain-containing protein 2 (LEMD2) cardiomyopathy in a subject in need thereof.

Another embodiment provides a knock-in mouse comprising T38>G mutation in the LEM domain-containing protein 2 (LEMD2). In certain aspects, said mutation is heterozygous. In some aspects, said mutation is homozyogous.

A further embodiment provides a method of making a transgenic mouse comprising contacting a murine cell with Cas9 and a single-stranded oligonucleotide (ssODN) template targeting LEM domain-containing protein 2 (LEMD2) nucleotide 38 within codon 13 of the coding region to result in replacement of thymine with guanine (G) (c.T38>G), yielding a leucine to arginine substitution in Lemd2.

Also provided is a method of making a knock-in mouse strain comprising contacting a murine cell with Cas9, guide RNA (gRNA) targeting LEM domain-containing protein 2 (LEMD2) sequence and a single-stranded oligonucleotide (ssODN) template containing a thymine to guanine (G) substitution in nucleotide 38 (c.T38>G) within codon 13 of the coding region of LEMD2, yielding a leucine to arginine substitution in Lemd2 mouse endogenous gene.

Other objects, features and advantages of the present invention will become apparent from the following detailed description. It should be understood, however, that the detailed description and the specific examples, while indicating preferred embodiments of the invention, are given by way of illustration only, since various changes and modifications within the spirit and scope of the invention will become apparent to those skilled in the art from this detailed description.

### BRIEF DESCRIPTION OF THE DRAWINGS

The following drawings form part of the present specification and are included to further demonstrate certain aspects of the present invention. The invention may be better understood by reference to one or more of these drawings in combination with the detailed description of specific embodiments presented herein.

FIGS. 1A-F. Cardiac abnormalities in Lemd2 KI/KI mice. (FIG. 1A) WT and Lemd2 c.T38>G (KI) alleles showing the sgRNA and the protospacer adjacent motif (PAM) sequences as well as the introduced pathogenic (red) and silent (green) mutations (SEQ ID NOs: 58-61). (FIG. 1B) Survival curve of WT (n=17), Lemd2 KI/+ (n=22) and Lemd2 KI/KI (n=15) mice (Log-rank (Mantel-Cox) test; ****p<0.0001 for WT versus Lemd2 KI/KI comparison). (FIG. 1C) Western blot showing the levels of two cardiac LEMD2 protein isoforms in 2-month-old WT (n=4) and Lemd2 KI/KI (n=4) mice. Bottom: Average and standard error mean (SEM) of the relative LEMD2/GAPDH densitometry ratio in WT and Lemd2 KI/KI mice. (FIG. 1D) Macroscopic images of hearts from 3-month-old WT and Lemd2 KI/KI mice (scale bar: 0.5 cm). (FIG. 1E) H&E staining of hearts of 3-month-old WT and Lemd2 KI/KI mice (scale bar: 500 m). (FIG. 1F) Masson's Trichrome staining of four-chamber view hearts from 3-month-old WT and Lemd2 KI/KI mice (scale bar: 500 μm).

FIGS. 2A-L. Lemd2 KI/KI mice develop systolic dysfunction and electrical abnormalities. (FIGS. 2A-F) Echocardiographic analysis of structural and functional parameters in systolic hearts from 2-month-old WT (n=7) and Lemd2 KI/KI (n=10) mice: (FIG. 2A) systolic left ventricular anterior wall (LVAW's) thickness, (FIG. 2B) systolic left ventricular internal diameter (LVID's), (FIG. 2C) ejection fraction (EF), (FIG. 2D) fractional shortening (FS) and (FIG. 2E) left ventricle volume (****p<0.0001, ***p<0.001; two-tailed unpaired t test). (FIG. 2F) Transthoracic M-mode echocardiographic tracings of 2-month-old mice WT and Lemd2 KI/KI. (FIGS. 2G-L) EGC analysis on 2-month-old WT (n=7) and Lemd2 KI/KI mice (n=10): (FIG. 2G) representative ECG of WT and Lemd2 KI/KI mice, (FIG. 2H) duration of P-R interval, (FIG. 2I) duration of QRS complex, (FIG. 2J) duration of the R-R interval, (FIG. 2K) heart rate and (FIG. 2L) duration of corrected QT (QTc) interval (P-R **p<0.01, QRS **p<0.01, R-R, Heart rate and QTc ns (non-significant) p>0.05; two-tailed unpaired t test).

FIGS. 3A-E. Chromatin and transcriptomic alterations in the Lemd2 KI/KI mice. (FIG. 3A) Percentage of cardiomyocyte nuclei with reduction in nuclear envelope-associated heterochromatin (n=4 mice per genotype; *p<0.05 two-tailed unpaired t test). (FIG. 3B) Representative electron microscopy pictures of 3-month-old WT and Lemd2 KI/KI cardiomyocyte nuclei (top scale bar: 2 m; bottom scale bar: 200 nm). (FIG. 3C) Heatmap showing the differentially expressed genes in hearts from WT and Lemd2 KI/KI mice (n=3 mice per genotype, expression level in Z-score, FDR-adjusted p-value (Benjamini-Hochberg method) of 0.05 was used as cutoff). (FIG. 3D) Volcano plot showing fold-change and p-value of genes up regulated (red) and down-regulated (blue) in hearts Lemd2 KI/KI compared with WT mice (n=3 mice per genotype, FDR-adjusted (Benjamini-Hochberg method) p-value<0.05 and fold-change>1.5). (FIG. 3E) GO terms up- and down-regulated in hearts from Lemd2 KI/KI mice compared to those from WT animals (n=3 mice per genotype, p<0.01).

FIGS. 4A-J. Cardiomyocyte hypertrophy and DNA damage in Lemd2 KI/KI mice. (FIG. 4A) Representative images of isolated cardiomyocytes from 3-month-old WT and Lemd2 KI/KI mice (scale bar: 50 μm). (FIG. 4B) Length of binucleated cardiomyocytes isolated from 3-month-old WT and Lemd2 KI/KI mice (n=3-4 mice per genotype, 100-150 total cells per genotype; ****p<0.0001 two-tailed unpaired t test). (FIG. 4C) Width of binucleated cardiomyocytes isolated from 3-month-old WT and Lemd2 KI/KI mice (n=3-4 mice per genotype, 100-150 total cells per genotype; ****p<0.0001 two-tailed unpaired t test). (FIG. 4D) Area of binucleated cardiomyocytes isolated from 3-month-old WT and Lemd2 KI/KI mice (n=3-4 mice per genotype, 100-150 total cells per genotype; ****p<0.0001 two-tailed unpaired t test). (FIG. 4E) GSEA plot showing the enrichment of genes related to genotoxic damage in hearts from Lemd2 KU/KI mice. Note that the enrichment score (green line) deviates from 0 in the right part of the plot, indicating that those genes are enriched in the Lemd2 KI/KI mice (3 mice per genotype). (FIG. 4F) Percentage of cardiac nuclei positive for γ-H2AX staining in WT and Lemd2 KI/KI mice (n=3-4 mice per genotype, more than 100 nuclei per mouse; *p<0.05 two-tailed unpaired t test). (FIG. 4G) Representative pictures of γ-H2AX and cTNT staining in cardiac sections from WT and Lemd2 KI/KI mice (scale bar: 20 μm). Note that the white square part of the bottom left panel has been zoomed in. (FIGS. 4H-J) Cardiac mRNA expression of genes related to p53 signaling and DNA damage response in WT and Lemd2 KI/KI (n=4 mice per genotype; Myc *p<0.05, Gadd45g *p<0.05, Scd1 *p<0.05 two-tailed unpaired t test).

FIGS. 5A-H. Lemd2 deficiency in the heart leads to cardiomyopathy and premature death in mice. (FIG. 5A) Representative picture of Lemd2^{fl/fl} (left) and cardiac-specific knock-out (cKO) (right) mice at P7 (scale bar: 1 cm). (FIG. 5B) Survival curve of Lemd2^{fl/fl} (n=19) and cardiac-specific knock-out (cKO) (n=23) mice. (Log-rank (Mantel-Cox) test; p<0.0001). (FIG. 5C) Echocardiographic analysis of systolic ejection fraction (EF) of hearts from Lemd2^{fl/fl} (n=7) and cardiac-specific knock-out (cKO) (n=3) mice (from 2 to 10 postnatal days, **p<0.01 two-tailed unpaired t test). (FIG. 5D) Echocardiographic analysis of systolic fractional shortening (FS) of hearts from Lemd2^{fl/fl} (n=7) and cardiac-specific knock-out (cKO) (n=3) mice (from 2 to 10 postnatal days; **p<0.01 two-tailed unpaired t test). (FIG. 5E) H&E staining of four-chamber view P7 hearts from Lemd2^{fl/fl} and cardiac-specific knock-out (cKO) mice (scale bar: 500 m). Note the atrial thrombi in cardiac-specific knock-out (cKO) hearts. (FIG. 5F) Heatmap showing the differentially expressed genes in hearts from Lemd2^{fl/fl} and cardiac-specific knock-out (cKO) mice (n=3 mice per genotype, expression level in Z-score, FDR-adjusted p-value (Benjamini-Hochberg method) of 0.05 was used as cutoff). (FIG. 5G) Enriched GO terms up- and down-regulated in hearts from cardiac-specific knock-out (cKO) compared to Lemd2^{fl/fl} mice (3 mice per genotype, p-value<0.01). (FIG. 5H) Upstream regulator analysis of differentially expressed genes in hearts from cardiac-specific knock-out (cKO) mice compared to those from Lemd2^{fl/fl} mice (n=4 mice per genotype, activation in Z-score).

FIGS. 6A-H. DNA damage and cellular apoptosis in Lemd2 cardiac-specific knock-out (cKO) mice. (FIG. 6A) Schematic representation of the confiner device. (FIG. 6B) Percentage of nuclei positive for γ-H2AX in Lemd2^{fl/fl} and cardiac-specific knock-out (cKO) cardiomyocytes, at baseline and after 20 μm compression for 1 hour, isolated from Pt mice (3 experimental replicates, n=2-5 mice per genotype and replicate, more than 80 nuclei per replicate, ***p<0.001, **p<0.01, *p<0.05; one-way ANOVA and Holm-Sidik test for correction of multiple comparisons). (FIG. 6C) Representative images of γ-H2AX and cTNT staining in cardiomyocytes, at baseline and after 20 m compression for 1 hour, isolated from P1 Lemd2^{fl/fl} and Lemd2 cardiac-specific knock-out (cKO) mice (scale bar: 20 μm). (FIG. 6D) Percentage of TUNEL positive nuclei in Lemd2^{fl/fl} and cardiac-specific knock-out (cKO) cardiomyocytes, at baseline and after 20 m compression for 1 hour, isolated from P1 mice (2-3 replicates, n=2-5 mice per genotype and replicate, more than 80 nuclei per replicate, **p<0.01, *p<0.05; one-way ANOVA and Holm-Sidik test for correction of multiple comparisons). (FIG. 6E) Representative images of TUNEL and cTnI staining in Lemd2^{fl/fl} and Lemd2 cardiac-specific knock-out (cKO) cardiomyocytes isolated from P1 mice and compressed at 20 m for 1 hour (scale bar: 20 μm). (FIG. 6F) Nuclei area in cardiomyocytes, at baseline and after 20 μm compression for 1 hour, isolated from P1 Lemd2^{fl/fl} and Lemd2 cardiac-specific knock-out (cKO) mice (n=2 mice per genotype, 15-30 nuclei per genotype, *p<0.05; one-way ANOVA and Holm-Sidik test for correction of multiple comparisons. (FIG. 6G) Nuclear solidity (area/convex area) in cardiomyocytes, at baseline and after 20 μm compression for 1 hour, isolated from P1 Lemd2^{fl/fl} and Lemd2 cardiac-specific knock-out (cKO) mice (n=2 mice per genotype, 15-30 nuclei per genotype, ***p<0.001, *p<0.05; one-way ANOVA and Holm-Sidik test for correction of multiple comparisons). (FIG. 6H) Representative pictures of lamin B1 and cTnI staining in Lemd2^{fl/fl} and Lemd2 cardiac-specific knock-out (cKO) cardiomyocytes isolated from P1 mice and compressed at 20 μm for 1 hour (scale bar: 5 μm).

FIGS. 7A-L. Lemd2 gene therapy improves cardiac function in Lemd2 KI/KI mice. (FIG. 7A) Schematic of the AAV9-Lemd2 system for in vivo delivery. (FIG. 7B) Overview of the in vivo injection strategy. (FIGS. 7C-G) Echocardiographic analysis of structural and functional parameters in hearts from 2-month-old WT (n=7), Lemd2 KI/KI (n=10) and KI/KI AAV9-Lemd2 mice (n=4) mice. The AAV9-Lemd2 treatment experiment was unblinded for mouse genotypes and data are compared to untreated WT and Lemd2 KI/KI groups shown in FIGS. 2A-F that were not assessed contemporaneously. ****p<0.0001, ***p<0.001 **p<0.01, *p<0.05; one-way ANOVA and Holm-Sidik test for correction of multiple comparisons. (FIG. 7C) systolic left ventricular anterior wall (LVAW's) thickness, (FIG. 7D) systolic left ventricular internal diameter (LVID's), (FIG. 7E) ejection fraction (EF), (FIG. 7F) fractional shortening (FS) and (FIG. 7G) left ventricle volume. (FIG. 7H) H&E staining of four-chamber view of 3-month-old hearts from WT, Lemd2 KI/KI and KI/KI AAV9-Lemd2 mice (scale bar: 500 m). (FIG. 7I) Masson Trichrome staining of 3-month-old hearts from WT, Lemd2 KI/KI and KI/KI AAV9-Lemd2 mice (scale bar: 50 m). (FIG. 7J) Quantification of the percentage of cardiac fibrosis in hearts from WT, Lemd2 KI/KI and KI/KI AAV9-Lemd2 mice (4-5 cardiac sections per mouse, n=1 mouse per genotype, **p<0.01, *p<0.05; one-way ANOVA and Holm-Sidik test for correction of multiple comparisons). (FIG. 7K) Lemd2 mRNA expression in hearts from 2-month-old WT (n=4), Lemd2 KI/KI (n=4) and KI/KI AAV9-Lemd2 (n=3) mice (***p<0.001; one-way ANOVA and Holm-Sidik test for correction of multiple comparisons). (FIG. 7L) Western blot showing the levels of two cardiac LEMD2 protein isoforms in 2-month-old WT (n=3), Lemd2 KI/KI (n=3) and KI/KI AAV9-Lemd2 (n=3) mice. Bottom: Average and standard error mean (SEM) of the relative LEMD2/GAPDH densitometry ratio in WT, KI/KI and KI/KI AAV9-Lemd2 mice.

FIG. 8. Survival curve of Lemd2 KI/KI (n=15) and Lemd2 KI/KI+AAV-9-Lemd2 (n=11) mice. **P<0.01, log-rank (Mantel-Cox) test.

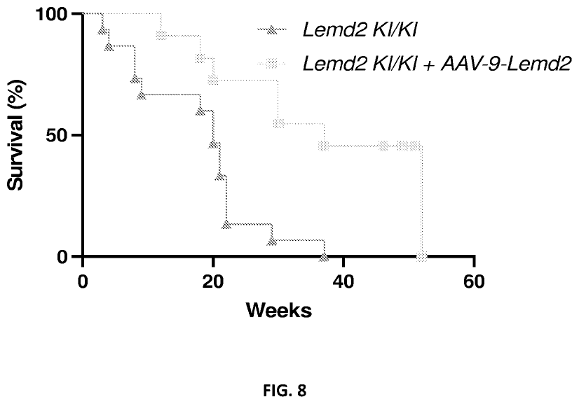

FIGS. 9A-J. Generation and characterization of the Lemd2 KI/KI mouse model. (FIG. 9A) Lemd2 mRNA expression in mouse tissues normalized to lung (GP, gastrocnemius-plantaris; TA, tibialis anterior; WAT, white adipose tissue). (FIG. 9B) Schematic of the CRISPR/Cas9 strategy to generate the Lemd2 KI/KI mice. (FIG. 9C) Sanger sequencing of a Lemd2 KU/KI mouse (SEQ ID NOs: 62 & 63). (FIG. 9D) Genotype frequency distribution seven days after birth (P7) of WT, heterozygous (KI/+) and homozygous Lemd2 c.T38>G (KI/KI) mice from heterozygous breeding (387 mice, p=non-significant; Chi-square test). (FIG. 9E) Lemd2 mRNA expression relative to Gapdh in hearts from 2-month-old WT (n=4) and Lemd2 KI/KI (n=4) mice (*p<0.05; two-tailed unpaired t test). (FIG. 9F) Immunofluorescence showing the localization of LEMD2 WT and LEMD2 c.T38>G after their retroviral overexpression in C2C12 myotubes (scale bar: 10 m). (FIG. 9G) Heart weight/tibia length ratio in WT and Lemd2 KI/KI mice (ns (non-significant) p>0.05; two-tailed unpaired t test). (FIG. 9H) Masson Trichrome staining of hearts from WT and Lemd2 KI/KI mice (scale bar: 50 μm). (FIG. 9I) H&E staining of tibialis anterior (TA) and gastrocnemius muscle groups from WT and Lemd2 KI/KI mice (scale bar: 50 μm). (FIG. 9J) Tibialis anterior (TA) weight/tibia length ratio in WT and Lemd2 KI/KI mice (ns (non-significant) p>0.05; two-tailed unpaired t test).

FIGS. 10A-D. Lemd2 KI/KI mice develop dilated cardiomyopathy (DCM). Echocardiographic analysis of structural and functional parameters in systolic hearts from WT and Lemd2 KI/KI mice: (FIG. 10A) Systolic left ventricular anterior wall (LVAW's) thickness (3 w **p<0.01, 4 w ****p<0.0001, 8 w ****p<0.0001, two-tailed unpaired student t test). (FIG. 10B) Systolic left ventricular internal diameter (LVID's) (3 w **p=0.01, 4 w ****p<0.0001, 8 w ****p<0.0001, two-tailed unpaired student t test), (FIG. 10C) Ejection fraction (EF) (3 w **p<0.01, 4 w ****p<0.0001, 8 w ****p<0.0001, two-tailed unpaired student t-test) and (FIG. 10D) Fractional shortening (FS) (3 w **p<0.01, 4 w ****p<0.0001, 8 w ****p<0.0001, 7 WT and 6 Lemd2 KI/KI mice for the 3 w comparison and 7 WT and 10 Lemd2 KI/KI mice for the 4 w and 8 w comparisons; two-tailed unpaired student t test).

FIGS. 11A-G. Mice heterozygous for the Lemd2 c.T38>G (KI/+) mutation have a preserved cardiac function. Echocardiographic analysis of structural and functional parameters in systolic hearts from 2-month-old WT (n=3) and Lemd2 KI/+ mice: (FIG. 11A) Systolic left ventricular anterior wall (LVAW's) thickness, (FIG. 11B) Systolic left ventricular internal diameter (LVID's), (FIG. 11C) Systolic left ventricular posterior wall (LVPW's) thickness, (FIG. 11D) Ejection fraction (EF), (FIG. 11E) Fractional shortening (FS) and (FIG. 11F) Left ventricle volume (n=6). (FIG. 11G) Representative transthoracic M-mode echocardiographic tracings of 2-month-old WT and Lemd2 KI/+ mice. (ns (non-significant) p>0.05; two-tailed unpaired t test for all the comparisons).

FIGS. 12A-E. Lemd2 KI/KI mice show cardiac electrical abnormalities. (FIG. 12A) ECG of two 2-month-old Lemd2 KI/KI mice showing the type II AV block (arrows indicate the absence of the QRS complex). (FIG. 12B) Schematic of the isoproterenol (ISO) administration protocol. (FIG. 12C) Representative ECG from 4/5-month-old mice before (basal) and after ISO administration (arrows indicate the absence of the QRS complex). (FIG. 12D) Survival curve of WT (n=3) and Lemd2 KI/KI (n=3) mice after the first ISO administration (Log-rank (Mantel-Cox) test; *p<0.05). (FIG. 12E) Immunostaining of cardiac sections from WT and Lemd2 KI/KI mice against the cardiomyocyte marker cardiac troponin T (cTnT) and the cardiac conduction system-specific marker HCN4. (White lines mark the AV node; RA: right atrium; scale bar: 100 μm).

FIGS. 13A-G. Structure, sarcomere contractility and calcium dynamics in Lemd2 KI/KI isolated cardiomyocytes. (FIG. 13A) Representative sarcomere contraction (top) and calcium transients (bottom) of WT and Lemd2 KI/KI cardiomyocytes. (FIG. 13B) Sarcomere length in cardiomyocytes from WT (n=4) and Lemd2 KI/KI (n=3) mice. (FIG. 13C) Percentage of sarcomere fractional shortening in cardiomyocytes from WT (n=4) and Lemd2 KI/KI (n=3) mice. (FIG. 13D) Ratio of diastolic calcium levels (F₃₄₀/F₃₅₀) measured using the Fura-2 dye in cardiomyocytes from WT (n=4) and Lemd2 KI/KI (n=3) mice. (FIG. 13E) Calcium transient amplitude (from basal level to peak) measured using the Fura-2 dye in cardiomyocytes from WT (n=4) and Lemd2 KI/KI (n=3) mice. (FIG. 13F) Time to calcium peak measured using the Fura-2 dye in cardiomyocytes from WT (n=4) and Lemd2 KI/KI (n=3) mice. (FIG. 13G) Representative transmission EM pictures of the sarcomere structure of 3-month-old WT and Lemd2 KI/KI hearts (scale bar: 2 μm). (ns (non-significant) p>0.05; two-tailed unpaired t test for all the comparisons).

FIGS. 14A-E. Generation and characterization of Lemd2 cardiac-specific knock-out (cKO) mice. (FIG. 14A) Scheme showing the Lemd2-floxed allele. (FIG. 14B) PCR showing the excision of the Lemd2-floxed allele (first exon) in cardiac tissue after the expression of the recombinase Cre under control of the αMHC promoter (floxed band=1.5 Kb; KO band=80 bp). (FIG. 14C) Western blot analysis showing the expression of both LEMD2 cardiac isoforms in heart protein lysates from Lemd2^{fl/fl} and cardiac-specific knock-out (cKO) mice. (FIG. 14D) Genotype frequency distribution one day after birth (P1) of Lemd2-floxed mice from Lemd2^{fl/fl} αMHC-Cre×Lemd2^{fl/fl} breeding (100 mice, p=non-significant; Chi-square test). (FIG. 14E) Transthoracic M-mode echocardiographic tracings of P7 Lemd2^{fl/fl} and cardiac-specific knock-out (cKO) mice.

FIGS. 15A-H. Activation of p53 signaling pathway, DNA damage and cellular apoptosis in Lemd2 cardiac-specific knock-out (cKO) mice. (FIG. 15A) GSEA plot showing the enrichment of genes related p53 downstream pathway in Lemd2 cardiac-specific knock-out (cKO) mice. Note that the enrichment score (green line) deviates from 0 in the right part of the plot, indicating that those genes are enriched in the Lemd2 cardiac-specific knock-out (cKO) mice (3 mice per genotype). (FIG. 15B) mRNA expression, normalized to 18S, of genes related to p53 signaling and DNA damage response (DDR) in WT and Lemd2 cardiac-specific knock-out (cKO) hearts (4 mice per genotype; Cdkn1a **p<0.01, Myc **p<0.01, Gadd45b *p<0.05, Btg2 **p<0.01, Atf3 *p<0.05, Bcl2 ***p<0.001, Gadd45g **p<0.01, Hmox1 *p<0.05, Sphk1 *p<0.05 and Bcl2111 ****p<0.0001; two-tailed unpaired t test). (FIG. 15C) Quantification of the percentage of nuclei positive for γ-H2AX staining in Lemd2^{fl/fl} and cardiac-specific knock-out (cKO) hearts (3-4 mice per genotype, more than 100 nuclei per mouse, **p<0.01 two-tailed unpaired t test). (FIG. 15D) Representative pictures of γ-H2AX staining in cardiac sections from P5 Lemd2^{fl/fl} and Lemd2 cardiac-specific knock-out (cKO) mice (scale bar: 20 m). (FIG. 15E) Quantification of the percentage of nuclei positive for Ki67 staining in Lemd2^{fl/fl} and cardiac-specific knock-out (cKO) hearts (3 mice per genotype, more than 500 nuclei per mouse, *p=0.048 two-tailed unpaired t test). (FIG. 15F) Representative pictures of Ki67 staining in cardiac sections from P5 Lemd2^{fl/fl} and Lemd2 cardiac-specific knock-out (cKO) mice (scale bar: 20 m). (FIG. 15G) Quantification of the nuclei positive for TUNEL staining in Lemd2^{fl/fl} and cardiac-specific knock-out (cKO) mice (3-4 mice per genotype, more than 100 nuclei per mouse, **p=0.0493 two-tailed unpaired t test). (FIG. 15H) Representative pictures of TUNEL staining in cardiac sections from P5 WT and Lemd2 cardiac-specific knock-out (cKO) mice (scale bar: 20 μm).

### DETAILED DESCRIPTION

In order to explore the role of LEMD2 in cardiac function and disease, the inventors generated a “humanized” Lemd2 knock-in (KI) mouse line, carrying the same c.T38>G homozygous mutation found in human patients, and showed that these animals develop systolic dysfunction and dilated cardiomyopathy (DCM) and die prematurely. They also showed that cardiomyocytes (CMs) isolated from homozygous KI animals (KI/KI) are hypertrophic and display a reduction in transcriptionally repressed heterochromatin associated with the nuclear envelope. Cardiac-specific Lemd2 knock-out (cKO) mice also die shortly after birth due to cardiac abnormalities. Transcriptomic analysis in these two mouse models revealed strong activation of the p53 pathway. The aberrant activation of p53, a master regulator of genome integrity, is caused by extensive DNA damage triggered by LEMD2 loss-of-function, and results in chronic activation of the DNA damage response (DDR) and apoptosis in Lemd2 mutant mice. Immunostaining of isolated cardiomyocytes lacking LEMD2 revealed nuclear deformations and abnormal mechanotransduction. The inventors also showed that therapeutic delivery of the WT Lemd2 specifically to CMs with adeno-associated virus (AAV) improved cardiac function of the Lemd2 KI/KI mice. These findings highlight the important role of LEMD2 in cardiac homeostasis and provide mechanistic insights into the basis of LEMD2-associated cardiomyopathy, as well as demonstrating the therapeutic potential of LEMD2 gene augmentation. These and other aspects of the disclosure are set out in detail below.

### I. LEMD2

LEM domain containing protein 2 (LEMD2), also known as LEM domain nuclear envelope protein, is a transmembrane protein of the inner nuclear membrane that is involved in nuclear structure organization (Brachner et al., 2005). It also plays a role in cell signaling and differentiation (Huber et al., 2009). A deduced 503-amino acid human protein, LEMD2 has a LAP2-emerin-MAN1 (LEM) and a lamin-interacting domain at its N terminus, followed by 2 transmembrane domains and a C-terminal MAN1-Src1 C-terminal (MSC) domain. Human and mouse LEMD2 proteins share 83% amino acid identity. It is widely expressed in human and mouse tissues examined. Analysis identified orthologs of LEMD2 in rat, dog, chicken, rhesus macaque and C. elegans. The LEMD2 gene maps to chromosome 6p21.31 and contains 9 exons.

Brachner et al. (2005) found that LEMD2 bound to lamins A and C (150330) and required association with A-type lamins for proper retention at the nuclear envelope in human and other mammalian cell lines. Loss of lamin A/C at the nuclear envelope destabilized LEMD2 and caused its relocalization from the inner nuclear membrane to the endoplasmic reticulum. Deletion analysis identified a region within the N terminus of LEMD2 that was required for interaction with the C-terminal region of lamin A/C. Overexpression of LEMD2 in HeLa cells or C2C12 mouse myoblasts led to accumulation of LEMD2 in punctate structures in the nucleus, with intrusions of the nuclear membrane, and recruitment of A-type lamins and their binding proteins. Overexpression also impeded cytokinesis, resulting in 2 to 4 daughter cells connected by long tubular structures. Brachner et al. (2005) concluded that LEMD2 has a function in membrane assembly and dynamic organization of the nuclear envelope during the cell cycle.

Huber et al. (2009) found that knockdown of Net25 (also known as Lemd2) or emerin in C2C12 mouse myoblast cells inhibited myogenic differentiation upon shift to differentiation medium. Knockdown of Net25 or emerin also resulted in elevated Erk1 (MAPK3; 601795) activation. Pharmacologic inhibition of Erk activation rescued myogenic differentiation in Net25- or emerin-knockdown cultures. Expression of human NET25 in mouse Net25 and emerin double-knockdown cultures also rescued differentiation, suggesting redundant roles for NET25 and emerin in C2C12 cell differentiation. Truncation analysis revealed that the N-terminal nucleoplasmic domain of NET25 was required for Erk1 activation.

Von Appen et al., (2020) showed that the ability of LEMD2 to condense on microtubules governs the activation of ESCRTs and coordinated spindle disassembly. The LEM motif of LEMD2 binds BAF (603811), conferring on LEMD2 an affinity for chromatin, while an adjacent low-complexity domain promotes LEMD2 phase separation. A proline-arginine-rich sequence within the low complexity domain binds to microtubules and targets condensation of LEMD2 to spindle microtubules that traverse the nascent nuclear envelope. Furthermore, the winged-helix domain of LEMD2 activates the ESCRT-II/ESCRT-III hybrid protein CHMP7 (611130) to form co-oligomeric rings. Disruption of these events in human cells prevented the recruitment of downstream ESCRTs, compromised spindle disassembly, and led to defects in nuclear integrity and DNA damage. The authors also proposed that during nuclear reassembly LEMD2 condenses into a liquid-like phase and coassembles with CHMP7 to form a macromolecular O-ring seal at the confluence between membranes, chromatin, and the spindle. The properties of LEMD2 that they described, and the homologous architectures of related inner nuclear membrane proteins, suggested that phase separation may contribute to other critical envelope functions, including interphase repair and chromatin organization.

### II. LEMD2-RELATED CARDIOMYOPATHY

Cardiomyopathies are a group of diseases that affect the heart muscle. Early on there may be few or no symptoms. As the disease worsens, shortness of breath, feeling tired, and swelling of the legs may occur, due to the onset of heart failure. An irregular heartbeat and fainting may occur. Those affected are at an increased risk of sudden cardiac death.

In 2015 cardiomyopathy and myocarditis affected 2.5 million people. Hypertrophic cardiomyopathy affects about 1 in 500 people while dilated cardiomyopathy affects 1 in 2,500. They resulted in 354,000 deaths up from 294,000 in 1990. Arrhythmogenic right ventricular dysplasia is more common in young people.

Types of cardiomyopathies include hypertrophic cardiomyopathy, dilated cardiomyopathy, restrictive cardiomyopathy, arrhythmogenic right ventricular dysplasia, and Takotsubo cardiomyopathy (broken heart syndrome). In hypertrophic cardiomyopathy the heart muscle enlarges and thickens. In dilated cardiomyopathy, the ventricles enlarge and weaken. In restrictive cardiomyopathy the ventricle stiffens.

In many cases, the cause cannot be determined. Hypertrophic cardiomyopathy is usually inherited, whereas dilated cardiomyopathy is inherited in about one third of cases. Dilated cardiomyopathy may also result from alcohol, heavy metals, coronary artery disease, cocaine use, and viral infections. Restrictive cardiomyopathy may be caused by amyloidosis, hemochromatosis, and some cancer treatments. Broken heart syndrome is caused by extreme emotional or physical stress.

Treatment depends on the type of cardiomyopathy and the severity of symptoms. Treatments may include lifestyle changes, medications, or surgery. Surgery may include a ventricular assist device or heart transplant.

One particular cardiomyopathy is LEMD2-related cardiomyopathy. LEMD2, a nuclear envelope protein, has been shown to play an important role in the pathogenesis of inherited dilated cardiomyopathy. LEMD2 mutation carriers develop arrhythmic cardiomyopathy with mild impairment of left ventricular systolic function but severe ventricular arrhythmias leading to sudden cardiac death. Affected cardiac tissue from a deceased patient and fibroblasts exhibit elongated nuclei with abnormal condensed heterochromatin at the periphery. The patient fibroblasts demonstrate cellular senescence and reduced proliferation capacity, which may suggest an involvement of LEM domain containing protein 2 in chromatin remodeling processes and premature aging.

### III. EXPRESSION VECTORS AND NUCLEIC ACID DELIVERY

In some embodiments of the disclosure, expression cassettes are employed to express a protein product, either for subsequent purification and delivery to a cell/subject, or for use directly in a genetic-based delivery approach. Provided herein are expression vectors which contain a nucleic acid encoding LEMD2.

Any type of vector, such as any of those described herein, may be used to deliver the coding region of LEMD2. In some embodiments, the vector is a lipid nanoparticle, such as a non-viral vector. In some embodiments, the vector is a viral vector. In some embodiments, the viral vector is a non-integrating viral vector (i.e., that does not insert sequence from the vector into a host chromosome). In some embodiments, the viral vector is an adeno-associated virus vector (AAV), a lentiviral vector, an integrase-deficient lentiviral vector, an adenoviral vector, a vaccinia viral vector, an alphaviral vector, or a herpes simplex viral vector. In some embodiments, the vector comprises a cardiomyocyte-specific promoter. In some embodiments, the cardiomyocyte-specific promoter is a cardiac troponin T (cTnT) promoter. In any of the foregoing embodiments, the vector may be an adeno-associated virus vector 9 (AAV9).

In some embodiments, expression of LEMD2 is performed in a cardiac cell. In some embodiments, expression is performed in induced pluripotent stem cells (iPSCs) or iPSC-derived cardiomyocytes (iPSC-CMs). In embodiments, the iPSCs cells are differentiated after transformation. For example, the iPSC cells may be differentiated into a cardiac cell after transformation. In embodiments, the iPSCs cells are differentiated into cardiac muscle cells. In embodiments, the iPSCs cells are differentiated into cardiomyocytes. iPSCs cells may be induced to differentiate according to methods known to those of skill in the art.

### A. Regulatory Elements

Expression requires that appropriate signals be provided in the vectors and include various regulatory elements such as enhancers/promoters from both viral and mammalian sources that drive expression of the genes of interest in cells. Elements designed to optimize messenger RNA stability and translatability in host cells also are defined. The conditions for the use of a number of dominant drug selection markers for establishing permanent, stable cell clones expressing the products are also provided, as is an element that links expression of the drug selection markers to expression of the polypeptide.

Throughout this application, the term “expression cassette” is meant to include any type of genetic construct containing a nucleic acid coding for a gene product in which part or all of the nucleic acid encoding sequence is capable of being transcribed and translated, i.e., is under the control of a promoter. A “promoter” refers to a DNA sequence recognized by the synthetic machinery of the cell, or introduced synthetic machinery, required to initiate the specific transcription of a gene. The phrase “under transcriptional control” means that the promoter is in the correct location and orientation in relation to the nucleic acid to control RNA polymerase initiation and expression of the gene. An “expression vector” is meant to include expression cassettes comprised in a genetic construct that is capable of replication, and thus including one or more of origins of replication, transcription termination signals, poly-A regions, selectable markers, and multipurpose cloning sites.

The term promoter will be used here to refer to a group of transcriptional control modules that are clustered around the initiation site for RNA polymerase II. Much of the thinking about how promoters are organized derives from analyses of several viral promoters, including those for the HSV thymidine kinase (tk) and SV40 early transcription units. These studies, augmented by more recent work, have shown that promoters are composed of discrete functional modules, each consisting of approximately 7-20 bp of DNA, and containing one or more recognition sites for transcriptional activator or repressor proteins.

At least one module in each promoter functions to position the start site for RNA synthesis. The best-known example of this is the TATA box, but in some promoters lacking a TATA box, such as the promoter for the mammalian terminal deoxynucleotidyl transferase gene and the promoter for the SV40 late genes, a discrete element overlying the start site itself helps to fix the place of initiation.

In some embodiments, the expression cassettes of the disclosure are expressed by a muscle-cell specific promoter. This muscle-cell specific promoter may be constitutively active or may be an inducible promoter.

Additional promoter elements regulate the frequency of transcriptional initiation. Typically, these are located in the region 30-110 bp upstream of the start site, although a number of promoters have recently been shown to contain functional elements downstream of the start site as well. The spacing between promoter elements frequently is flexible, so that promoter function is preserved when elements are inverted or moved relative to one another. In the tk promoter, the spacing between promoter elements can be increased to 50 bp apart before activity begins to decline. Depending on the promoter, it appears that individual elements can function either co-operatively or independently to activate transcription.

In certain embodiments, viral promotes such as the human cytomegalovirus (CMV) immediate early gene promoter, the SV40 early promoter, the Rous sarcoma virus long terminal repeat, rat insulin promoter and glyceraldehyde-3-phosphate dehydrogenase can be used to obtain high-level expression of the coding sequence of interest. The use of other viral or mammalian cellular or bacterial phage promoters which are well-known in the art to achieve expression of a coding sequence of interest is contemplated as well, provided that the levels of expression are sufficient for a given purpose. By employing a promoter with well-known properties, the level and pattern of expression of the protein of interest following transfection or transformation can be optimized. Further, selection of a promoter that is regulated in response to specific physiologic signals can permit inducible expression of the gene product.

Enhancers are genetic elements that increase transcription from a promoter located at a distant position on the same molecule of DNA. Enhancers are organized much like promoters. That is, they are composed of many individual elements, each of which binds to one or more transcriptional proteins. The basic distinction between enhancers and promoters is operational. An enhancer region as a whole must be able to stimulate transcription at a distance; this need not be true of a promoter region or its component elements. On the other hand, a promoter must have one or more elements that direct initiation of RNA synthesis at a particular site and in a particular orientation, whereas enhancers lack these specificities. Promoters and enhancers are often overlapping and contiguous, often seeming to have a very similar modular organization.

Below is a list of promoters/enhancers and inducible promoters/enhancers that could be used in combination with the nucleic acid encoding a gene of interest in an expression construct. Additionally, any promoter/enhancer combination (as per the Eukaryotic Promoter Data Base EPDB) could also be used to drive expression of the gene. Eukaryotic cells can support cytoplasmic transcription from certain bacterial promoters if the appropriate bacterial polymerase is provided, either as part of the delivery complex or as an additional genetic expression construct.

The promoter and/or enhancer may be, for example, immunoglobulin light chain, immunoglobulin heavy chain, T-cell receptor, HLA DQ a and/or DQ β, β-interferon, interleukin-2, interleukin-2 receptor, MHC class II 5, MHC class II HLA-Dra, β-Actin, muscle creatine kinase (MCK), prealbumin (transthyretin), elastase I, metallothionein (MTII), collagenase, albumin, α-fetoprotein, t-globin, β-globin, c-fos, c-HA-ras, insulin, neural cell adhesion molecule (NCAM), α₁-antitrypain, H2B (TH2B) histone, mouse and/or type I collagen, glucose-regulated proteins (GRP94 and GRP78), rat growth hormone, human serum amyloid A (SAA), troponin I (TN I), platelet-derived growth factor (PDGF), duchenne muscular dystrophy, SV40, polyoma, retroviruses, papilloma virus, hepatitis B virus, human immunodeficiency virus, cytomegalovirus (CMV), and gibbon ape leukemia virus.

In some embodiments, inducible elements may be used. In some embodiments, the inducible element is, for example, MTII, MMTV (mouse mammary tumor virus), β-interferon, adenovirus 5 E2, collagenase, stromelysin, SV40, murine MX gene, GRP78 gene, α-2-macroglobulin, vimentin, MHC class I gene H-2κb, HSP70, proliferin, tumor necrosis factor, and/or thyroid stimulating hormone a gene. In some embodiments, the inducer is phorbol ester (TFA), heavy metals, glucocorticoids, poly(rI)x, poly(rc), ElA, phorbol ester (TPA), interferon, Newcastle Disease Virus, A23187, IL-6, serum, interferon, SV40 large T antigen, PMA, and/or thyroid hormone. Any of the inducible elements described herein may be used with any of the inducers described herein.

Of particular interest are cardiomyocyte-specific promoters. In some embodiments, the cardiomyocyte-specific promoter is the cardiac troponin T (cTnT) promoter.

Where a cDNA insert is employed, one will typically desire to include a polyadenylation signal to effect proper polyadenylation of the gene transcript. Any polyadenylation sequence may be employed such as human growth hormone and SV40 polyadenylation signals. Also contemplated as an element of the expression cassette is a terminator. These elements can serve to enhance message levels and to minimize read through from the cassette into other sequences.

### B. Delivery of Expression Vectors

In addition to viral delivery employing the viral vectors mentioned above, several non-viral methods for the transfer of expression constructs into cultured mammalian cells also are contemplated by the present disclosure. These include calcium phosphate precipitation, DEAE-dextran, electroporation, direct microinjection, DNA-loaded liposomes and lipofectamine-DNA complexes, cell sonication, gene bombardment using high velocity microprojectiles, and receptor-mediated transfection. Some of these techniques may be successfully adapted for in vivo or ex vivo use.

Once the expression construct has been delivered into the cell the nucleic acid encoding the gene of interest may be positioned and expressed at different sites. In certain embodiments, the nucleic acid encoding the gene may be stably integrated into the genome of the cell. This integration may be in the cognate location and orientation via homologous recombination (gene replacement), or it may be integrated in a random, non-specific location (gene augmentation). In yet further embodiments, the nucleic acid may be stably maintained in the cell as a separate, episomal segment of DNA. Such nucleic acid segments or “episomes” encode sequences sufficient to permit maintenance and replication independent of or in synchronization with the host cell cycle. How the expression construct is delivered to a cell and where in the cell the nucleic acid remains is dependent on the type of expression construct employed.

In yet another embodiment, the expression construct may simply consist of naked recombinant DNA or plasmids. Transfer of the construct may be performed by any of the methods mentioned above which physically or chemically permeabilize the cell membrane. This is particularly applicable for transfer in vitro but it may be applied to in vivo use as well. DNA encoding a gene of interest may also be transferred in a similar manner in vivo and express the gene product.

In still another embodiment for transferring a naked DNA expression construct into cells may involve particle bombardment. This method depends on the ability to accelerate DNA-coated microprojectiles to a high velocity allowing them to pierce cell membranes and enter cells without killing them. Several devices for accelerating small particles have been developed. One such device relies on a high voltage discharge to generate an electrical current, which in turn provides the motive force. The microprojectiles used have consisted of biologically inert substances such as tungsten or gold beads.

In some embodiments, the expression construct is delivered directly to the liver, skin, and/or muscle tissue of a subject. This may require surgical exposure of the tissue or cells, to eliminate any intervening tissue between the gun and the target organ, i.e., ex vivo treatment. Again, DNA encoding a particular gene may be delivered via this method and still be incorporated by the present disclosure.

In a further embodiment, the expression construct may be entrapped in a liposome. Liposomes are vesicular structures characterized by a phospholipid bilayer membrane and an inner aqueous medium. Multilamellar liposomes have multiple lipid layers separated by aqueous medium. They form spontaneously when phospholipids are suspended in an excess of aqueous solution. The lipid components undergo self-rearrangement before the formation of closed structures and entrap water and dissolved solutes between the lipid bilayers. Also contemplated are lipofectamine-DNA complexes.

Liposome-mediated nucleic acid delivery and expression of foreign DNA in vitro has been very successful. A reagent known as Lipofectamine 2000™ is widely used and commercially available.

In certain embodiments, the liposome may be complexed with a hemagglutinating virus (HVJ) to facilitate fusion with the cell membrane and promote cell entry of liposome-encapsulated DNA. In other embodiments, the liposome may be complexed or employed in conjunction with nuclear non-histone chromosomal proteins (HMG-1). In yet further embodiments, the liposome may be complexed or employed in conjunction with both HVJ and HMG-1. In that such expression constructs have been successfully employed in transfer and expression of nucleic acid in vitro and in vivo, then they are applicable for the present disclosure. Where a bacterial promoter is employed in the DNA construct, it also will be desirable to include within the liposome an appropriate bacterial polymerase.

Other expression constructs which can be employed to deliver a nucleic acid encoding a particular gene into cells are receptor-mediated delivery vehicles. These take advantage of the selective uptake of macromolecules by receptor-mediated endocytosis in almost all eukaryotic cells. Because of the cell type-specific distribution of various receptors, the delivery can be highly specific.

Receptor-mediated gene targeting vehicles generally consist of two components: a cell receptor-specific ligand and a DNA-binding agent. Several ligands have been used for receptor-mediated gene transfer. The most extensively characterized ligands are asialoorosomucoid (AS OR) and transferrin. A synthetic neoglycoprotein, which recognizes the same receptor as ASOR, has been used as a gene delivery vehicle and epidermal growth factor (EGF) has also been used to deliver genes to squamous carcinoma cells.

### C. AAV Vectors

In embodiments, particular embodiments, the vector is an AAV vector. AAV is a small virus that infects humans and some other primate species. AAV is not currently known to cause disease. The virus causes a very mild immune response, lending further support to its apparent lack of pathogenicity. In many cases, AAV vectors integrate into the host cell genome, which can be important for certain applications, but can also have unwanted consequences. Gene therapy vectors using AAV can infect both dividing and quiescent cells and persist in an extrachromosomal state without integrating into the genome of the host cell, although in the native virus some integration of virally carried genes into the host genome does occur. These features make AAV a very attractive candidate for creating viral vectors for gene therapy, and for the creation of isogenic human disease models. Recent human clinical trials using AAV for gene therapy in the retina have shown promise. AAV belongs to the genus Dependoparvovirus, which in turn belongs to the family Parvoviridae. The virus is a small (20 nm) replication-defective, nonenveloped virus.

Wild-type AAV has attracted considerable interest from gene therapy researchers due to a number of features. Chief amongst these is the virus's apparent lack of pathogenicity. It can also infect non-dividing cells and has the ability to stably integrate into the host cell genome at a specific site (designated AAVS1) in the human chromosome 19. This feature makes it somewhat more predictable than retroviruses, which present the threat of a random insertion and of mutagenesis, which is sometimes followed by development of a cancer. The AAV genome integrates most frequently into the site mentioned, while random incorporations into the genome take place with a negligible frequency. Development of AAVs as gene therapy vectors, however, has eliminated this integrative capacity by removal of the rep and cap from the DNA of the vector. The desired gene together with a promoter to drive transcription of the gene is inserted between the inverted terminal repeats (ITR) that aid in concatemer formation in the nucleus after the single-stranded vector DNA is converted by host cell DNA polymerase complexes into double-stranded DNA. AAV-based gene therapy vectors form episomal concatemers in the host cell nucleus. In non-dividing cells, these concatemers remain intact for the life of the host cell. In dividing cells, AAV DNA is lost through cell division, since the episomal DNA is not replicated along with the host cell DNA. Random integration of AAV DNA into the host genome is detectable but occurs at very low frequency. AAVs also present very low immunogenicity, seemingly restricted to generation of neutralizing antibodies, while they induce no clearly defined cytotoxic response. This feature, along with the ability to infect quiescent cells present their dominance over adenoviruses as vectors for human gene therapy.

Use of the AAV does present some disadvantages. The cloning capacity of the vector is relatively limited and most therapeutic genes require the complete replacement of the virus's 4.8 kilobase genome. Large genes are, therefore, not suitable for use in a standard AAV vector. Options are currently being explored to overcome the limited coding capacity. The AAV ITRs of two genomes can anneal to form head to tail concatemers, almost doubling the capacity of the vector. Insertion of splice sites allows for the removal of the ITRs from the transcript.

Because of AAV's specialized gene therapy advantages, researchers have created an altered version of AAV termed self-complementary adeno-associated virus (scAAV). Whereas AAV packages a single strand of DNA and must wait for its second strand to be synthesized, scAAV packages two shorter strands that are complementary to each other. By avoiding second-strand synthesis, scAAV can express more quickly, although as a caveat, scAAV can only encode half of the already limited capacity of AAV. Recent reports suggest that scAAV vectors are more immunogenic than single stranded adenovirus vectors, inducing a stronger activation of cytotoxic T lymphocytes.

The humoral immunity instigated by infection with the wild-type is thought to be a very common event. The associated neutralising activity limits the usefulness of the most commonly used serotype AAV2 in certain applications. Accordingly, the majority of clinical trials currently under way involve delivery of AAV2 into the brain, a relatively immunologically privileged organ. In the brain, AAV2 is strongly neuron-specific.

The AAV genome is built of single-stranded deoxyribonucleic acid (ssDNA), either positive- or negative-sensed, which is about 4.7 kilobase long. The genome comprises inverted terminal repeats (ITRs) at both ends of the DNA strand, and two open reading frames (ORFs): rep and cap. The former is composed of four overlapping genes encoding Rep proteins required for the AAV life cycle, and the latter contains overlapping nucleotide sequences of capsid proteins: VP1, VP2 and VP3, which interact together to form a capsid of an icosahedral symmetry.

The Inverted Terminal Repeat (ITR) sequences comprise 145 bases each. They were named so because of their symmetry, which was shown to be required for efficient multiplication of the AAV genome. The feature of these sequences that gives them this property is their ability to form a hairpin, which contributes to so-called self-priming that allows primase-independent synthesis of the second DNA strand. The ITRs were also shown to be required for both integration of the AAV DNA into the host cell genome (19^{th} chromosome in humans) and rescue from it, as well as for efficient encapsidation of the AAV DNA combined with generation of a fully assembled, deoxyribonuclease-resistant AAV particles.

With regard to gene therapy, ITRs seem to be the only sequences required in cis next to the therapeutic gene: structural (cap) and packaging (rep) proteins can be delivered in trans. With this assumption many methods were established for efficient production of recombinant AAV (rAAV) vectors containing a reporter or therapeutic gene. However, it was also published that the ITRs are not the only elements required in cis for the effective replication and encapsidation. A few research groups have identified a sequence designated cis-acting Rep-dependent element (CARE) inside the coding sequence of the rep gene. CARE was shown to augment the replication and encapsidation when present in cis.

On the “left side” of the genome there are two promoters called p5 and p19, from which two overlapping messenger ribonucleic acids (mRNAs) of different lengths can be produced. Each of these contains an intron which can be either spliced out or not. Given these possibilities, four various mRNAs, and consequently four various Rep proteins with overlapping sequence can be synthesized. Their names depict their sizes in kilodaltons (kDa): Rep78, Rep68, Rep52 and Rep40. Rep78 and 68 can specifically bind the hairpin formed by the ITR in the self-priming act and cleave at a specific region, designated terminal resolution site, within the hairpin. They were also shown to be necessary for the AAVS1-specific integration of the AAV genome. All four Rep proteins were shown to bind ATP and to possess helicase activity. It was also shown that they upregulate the transcription from the p40 promoter (mentioned below) but downregulate both p5 and p19 promoters.

The right side of a positive-sensed AAV genome encodes overlapping sequences of three capsid proteins, VP1, VP2 and VP3, which start from one promoter, designated p40. The molecular weights of these proteins are 87, 72 and 62 kiloDaltons, respectively. The AAV capsid is composed of a mixture of VP1, VP2, and VP3 totaling 60 monomers arranged in icosahedral symmetry in a ratio of 1:1:10, with an estimated size of 3.9 MegaDaltons.

The cap gene produces an additional, non-structural protein called the Assembly-Activating Protein (AAP). This protein is produced from ORF2 and is essential for the capsid-assembly process. The exact function of this protein in the assembly process and its structure have not been solved to date.

All three VPs are translated from one mRNA. After this mRNA is synthesized, it can be spliced in two different manners: either a longer or shorter intron can be excised resulting in the formation of two pools of mRNAs: a 2.3 kb- and a 2.6 kb-long mRNA pool. Usually, especially in the presence of adenovirus, the longer intron is preferred, so the 2.3-kb-long mRNA represents the so-called “major splice”. In this form the first AUG codon, from which the synthesis of VP1 protein starts, is cut out, resulting in a reduced overall level of VP1 protein synthesis. The first AUG codon that remains in the major splice is the initiation codon for VP3 protein. However, upstream of that codon in the same open reading frame lies an ACG sequence (encoding threonine) which is surrounded by an optimal Kozak context. This contributes to a low level of synthesis of VP2 protein, which is actually VP3 protein with additional N terminal residues, as is VP1.

Since the bigger intron is preferred to be spliced out, and since in the major splice the ACG codon is a much weaker translation initiation signal, the ratio at which the AAV structural proteins are synthesized in vivo is about 1:1:20, which is the same as in the mature virus particle. The unique fragment at the N terminus of VP1 protein was shown to possess the phospholipase A2 (PLA2) activity, which is probably required for the releasing of AAV particles from late endosomes. VP2 and VP3 are crucial for correct virion assembly. More recently VP2 has been shown to be unnecessary for the complete virus particle formation and an efficient infectivity, and also presented that VP2 can tolerate large insertions in its N terminus, while VP1 cannot, probably because of the PLA2 domain presence.

In some embodiments, the AAV vector is an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of U.S. Patent Publication No. 2015/0111955, which is incorporated by reference herein in its entirety), AAV9 vector, AAV9P vector (also known as AAVMYO, see, Weinmann et al., 2020), Myo-AAV vectors described in Tabebordbar et al., 2021, (e.g., MyoAAV 1A, 2A, 3A, 4A, 4C, or 4E), and AAV9-rh74-HB-PI, AAV9-AAA-PI-SG vectors described in WO2022053630, wherein the number following AAV indicates the AAV serotype. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., 2001; Naso et al., 2017, and references cited therein for detailed discussion of various AAV vectors. In some embodiments, the vector is an AAV9 vector.

In some embodiments, the coding region for LEMD2 may be packaged into an AAV vector. In some embodiments, the AAV vector is a wild-type AAV vector. In some embodiments, the AAV vector contains one or more mutations. In some embodiments, the AAV vector is isolated or derived from an AAV vector of serotype AAV1, AAV2, AAV3, AAV4, AAV5, AAV6, AAV7, AAV8, AAV9, AAV10, AAV11 or any combination thereof.

Exemplary AAV vectors contain two ITR (inverted terminal repeat) sequences which flank a central sequence region comprising the LEMD2 coding sequence. In some embodiments, the ITRs are isolated or derived from an AAV vector of serotype AAV1, AAV2, AAV3, AAV4, AAV5, AAV6, AAV7, AAV8, AAV9, AAV10, AAV11 or any combination thereof. In some embodiments, the ITRs comprise or consist of full-length and/or wild-type sequences for an AAV serotype. In some embodiments, the ITRs comprise or consist of truncated sequences for an AAV serotype. In some embodiments, the ITRs comprise or consist of elongated sequences for an AAV serotype. In some embodiments, the ITRs comprise or consist of sequences comprising a sequence variation compared to a wild-type sequence for the same AAV serotype. In some embodiments, the sequence variation comprises one or more of a substitution, deletion, insertion, inversion, or transposition. In some embodiments, the ITRs comprise or consist of at least 100, 101, 102, 103, 104, 105, 106, 107, 108, 109, 110, 111, 112, 113, 114, 115, 116, 117, 118, 119, 120, 121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs comprise or consist of 100, 101, 102, 103, 104, 105, 106, 107, 108, 109, 110, 111, 112, 113, 114, 115, 116, 117, 118, 119, 120,121, 122, 123, 124, 125, 126, 127, 128, 129, 130, 131, 132, 133, 134, 135, 136, 137, 138, 139, 140, 141, 142, 143, 144, 145, 146, 147, 148, 149 or 150 base pairs. In some embodiments, the ITRs have a length of 110±10 base pairs. In some embodiments, the ITRs have a length of 120±10 base pairs. In some embodiments, the ITRs have a length of 130±10 base pairs. In some embodiments, the ITRs have a length of 140±10 base pairs. In some embodiments, the ITRs have a length of 150±10 base pairs. In some embodiments, the ITRs have a length of 115, 145, or 141 base pairs.

In some embodiments, the AAV vector may contain one or more nuclear localization signals (NLS). In some embodiments, the AAV vector contains 1, 2, 3, 4, or 5 nuclear localization signals. Exemplary NLS include the c-myc NLS, the SV40 NLS, the hnRNPAI M9 NLS, the nucleoplasmin NLS, the sequence RMRKFKNKGKDTAELRRRRVEVSVELRKAKKDEQILKRRNV (SEQ ID NO: 39) of the IBB domain from importin-alpha, the sequences VSRKRPRP (SEQ ID NO: 40) and PPKKARED (SEQ ID NO: 56) of the myoma T protein, the sequence PQPKKKPL (SEQ ID NO: 41) of human p53, the sequence SALIKKKKKMAP (SEQ ID NO: 42) of mouse c-abl IV, the sequences DRLRR (SEQ ID NO: 57) and PKQKKRK (SEQ ID NO: 43) of the influenza virus NS1, the sequence RKLKKKIKKL (SEQ ID NO: 44) of the Hepatitis virus delta antigen and the sequence REKKKFLKRR (SEQ ID NO: 45) of the mouse Mx1 protein. Further acceptable nuclear localization signals include bipartite nuclear localization sequences such as the sequence KRKGDEVDGVDEVAKKKSKK (SEQ ID NO: 46) of the human poly(ADP-ribose) polymerase or the sequence RKCLQAGMNLEARKTKK (SEQ ID NO: 47) of the steroid hormone receptors (human) glucocorticoid.

In some embodiments, the AAV vector may comprise additional elements to facilitate packaging of the vector and expression of the LEMD2. In some embodiments, the AAV vector may comprise a polyA sequence. In some embodiments, the polyA sequence may be a mini-polyA sequence. In some embodiments, the AAV vector may comprise a transposable element. In some embodiments, the AAV vector may comprise a regulator element. In some embodiments, the regulator element is an activator or a repressor.

In some embodiments, the AAV may contain one or more promoters. In some embodiments, the one or more promoters drive expression of LEMD2. In some embodiments, the one or more promoters are cardiomyocyte-specific promoters. Exemplary cardiac-specific promoters include the cardiac troponin T promoter and α-myosin heavy chain promoter.

In some embodiments, the AAV vector may be optimized for production in yeast, bacteria, insect cells, or mammalian cells. In some embodiments, the AAV vector may be optimized for expression in human cells. In some embodiments, the AAV vector may be optimized for expression in a bacculovirus expression system.

In some embodiments, the construct comprising a promoter and a nuclease further comprises at least two inverted terminal repeat (ITR) sequences. In some embodiments, the construct comprising a promoter and a nuclease further comprises at least two ITR sequences from isolated or derived from an AAV of serotype 2 (AAV9). In some embodiments, the construct comprises or consists of, from 5′ to 3′ a first ITR, a sequence encoding a promoter, a sequence encoding a nuclease and a second ITR. In some embodiments, the construct comprises or consists of, from 5′ to 3′ a first AAV9 ITR, a sequence encoding an cTnT promoter, a sequence encoding LEMD2, a SV40 poly(A) signal, a β-globin poly(A) signal, and a second AAV9 ITR. In some embodiments, the construct comprising or consisting of, from 5′ to 3′ a first ITR, a sequence encoding a promoter, a sequence encoding a nuclease and a second ITR, further comprises a poly A sequence. In some embodiments, the polyA sequence comprises or consists of a minipolyA sequence. Exemplary minipolyA sequences of the disclosure comprise or consist of a nucleotide sequence of TAGCAATAAAGGATCGTTTATTTTCATTGGAAGCGTGTGTTGGTTTTTTGATCAGGCGCG (SEQ ID NO: 48). In some embodiments, the construct comprises or consists of, from 5′ to 3′ a first ITR, a sequence encoding a promoter, a sequence encoding a nuclease, a poly A sequence and a second ITR. In some embodiments, the construct comprises or consists of, from 5′ to 3′ a first ITR, a sequence encoding a promoter, a sequence encoding a nuclease, a minipoly A sequence and a second ITR. In some embodiments, the construct comprises or consists of, from 5′ to 3′ a first AAV9 ITR, a sequence encoding an cTnT promoter, a sequence encoding a LEMD2 coding region, a minipoly A sequence and a second AAV9 ITR. In some embodiments, the construct comprising, from 5′ to 3′ a first ITR, a sequence encoding a promoter, a sequence encoding a nuclease, a poly A sequence and a second ITR, further comprises at least one nuclear localization signal. In some embodiments, the construct comprising, from 5′ to 3′ a first ITR, a sequence encoding a promoter, a sequence encoding a nuclease, a poly A sequence and a second ITR, further comprises at least two nuclear localization signals. Exemplary nuclear localization signals of the disclosure comprise or consist of a nucleotide sequence of AAGCGTCCTGCTGCTACTAAGAAAGCTGGTCAAGCTAAGAAAAAGAAA (SEQ ID NO: 49) or a nucleotide sequence of ATGGCCCCAAAGAAGAAGCGGAAGGTCGGTATCCACGGAGTCCCAGCAGCC (SEQ ID NO: 50). In some embodiments, the construct comprises or consists of, from 5′ to 3′ a first ITR, a sequence encoding a promoter, a sequence encoding a first nuclear localization signal, a sequence encoding a nuclease, a poly A sequence and a second ITR. In some embodiments, the construct comprises or consists of, from 5′ to 3′ a first ITR, a sequence encoding a promoter, a sequence encoding a first nuclear localization signal, a sequence encoding a nuclease, a sequence encoding a second nuclear localization signal, a poly A sequence and a second ITR. In some embodiments, the construct comprising, from 5′ to 3′ a first ITR, a sequence encoding a promoter, a sequence encoding a first nuclear localization signal, a sequence encoding a nuclease, a sequence encoding a second nuclear localization signal, a poly A sequence and a second ITR, further comprises a stop codon. The stop codon may have a sequence of TAG, TAA, or TGA. In some embodiments, the construct comprises or consists of, from 5′ to 3′ a first ITR, a sequence encoding a promoter, a sequence encoding a first nuclear localization signal, a sequence encoding a nuclease, a sequence encoding a second nuclear localization signal, a stop codon, a poly A sequence and a second ITR. In some embodiments, the construct comprising or consisting of, from 5′ to 3′ a first ITR, a sequence encoding a promoter, a sequence encoding a first nuclear localization signal, a sequence encoding a nuclease, a sequence encoding a second nuclear localization signal, a stop codon, a poly A sequence and a second ITR, further comprises transposable element inverted repeats. Exemplary regulatory sequences of the disclosure comprise or consist of a nucleotide sequence of CATGCAAGCTGTAGCCAACCACTAGAACTATAGCTAGAGTCCTGGGCGAACAAACGATGCTC GCCTTCCAGAAAACCGAGGATGCGAACCACTTCATCCGGGGTCAGCACCACCGGCAAGCGCC GCGACGGCCGAGGTCTTCCGATCTCCTGAAGCCAGGGCAGATCCGTGCACAGCACCTTGCCG TAGAAGAACAGCAAGGCCGCCAATGCCTGACGATGCGTGGAGACCGAAACCTTGCGCTCGTT CGCCAGCCAGGACAGAAATGCCTCGACTTCGCTGCTGCCCAAGGTTGCCGGGTGACGCACAC CGTGGAAAC GGATGAAGGCACGAACCCAGTTGACATAAGCCTGTTCGGTTTCGTAAACTGTAA TGCAAGTAGCGTATGCGCTCACGCAACTGGTCCAGAACCTTGACCGAACGCAGCGGTGGTAA CGGCGCAGTGGCGGTTTTCATGGCTTGTTATGACTGTTTTTTTGTACAGTCTATGCCTCGGG CATCCAAGCAGCAAGCGCGTTACGCCGTGGGTCGATGTTTGATGTTATGGAGCAGCAACGAT GTTACGCAGCAGCAACGATGTTACGCAGCAGGGCAGTCGCCCTAAAACAAAGTTAGGTGGCT CAAGTATGGGCATCATTCGCACATGTAGGCTCGGCCCTGACCAAGTCAAATCCATGCGGGCT GCTCTTGATCTTTTCGGTCGTGAGTTCGGAGACGTAGCCACCTACTCCCAACATCAGCCGGA CTCCGATTACCTCGGGAACTTGCTCCGTAGTAAGACATTCATCGCGCTTGCTGCCTTCGACC AAGAAGCGGTTGTTGGCGCTCTCGCGGCTTACGTTCTGCCCAAGTTTGAGCAGCCGCGTAGT GAGATCTATATCTATGATCTCGCAGTCTCCGGCGAGCACCGGAGGCAGGGCATTGCCACCGC GCTCATCAATCTCCTCAAGCATGAGGCCAACGCGCTTGGTGCTTATGTGATCTACGTGCAAG CAGATTACGGTGACGATCCCGCAGTGGCTCTCTATACAAAGTTGGGCATACGGGAAGAAGTG ATGCACTTTGATATCGACCCAAGTACCGCCACCTAACAATTCGTTCAAGCCGAGATCGGCTT CCCGGCCGCGGAGTTGTTCGGTAAATTGTCACAACGCCG (SEQ ID NO: 51). In some embodiments, the construct comprises or consists of, from 5′ to 3′ a first transposable element inverted repeat, a first ITR, a sequence encoding a promoter, a sequence encoding a first nuclear localization signal, a sequence encoding a nuclease, a sequence encoding a second nuclear localization signal, a stop codon, a poly A sequence, a second ITR, a regulatory sequence and a second transposable element inverted repeat. In some embodiments, the construct may further comprise one or more spacer sequences. Exemplary spacer sequences of the disclosure have length from 1-1500 nucleotides, inclusive of all ranges therebetween. In some embodiments, the spacer sequences may be located either 5′ to or 3′ to an ITR, a promoter, a nuclear localization sequence, a nuclease, a stop codon, a polyA sequence, a transposable element inverted repeat, and/or a regulator element.

### IV. PHARMACEUTICAL COMPOSITIONS AND DELIVERY METHODS

For clinical applications, pharmaceutical compositions are prepared in a form appropriate for the intended application. Generally, this entails preparing compositions that are essentially free of pyrogens, as well as other impurities that could be harmful to humans or animals.

Appropriate salts and buffers are used to render drugs, proteins or delivery vectors stable and allow for uptake by target cells. Aqueous compositions of the present disclosure comprise an effective amount of the drug, vector or proteins, dissolved or dispersed in a pharmaceutically acceptable carrier or aqueous medium. The phrase “pharmaceutically or pharmacologically acceptable” refer to molecular entities and compositions that do not produce adverse, allergic, or other untoward reactions when administered to an animal or a human. As used herein, “pharmaceutically acceptable carrier” includes solvents, buffers, solutions, dispersion media, coatings, antibacterial and antifungal agents, isotonic and absorption delaying agents and the like acceptable for use in formulating pharmaceuticals, such as pharmaceuticals suitable for administration to humans. The use of such media and agents for pharmaceutically active substances is well known in the art. Any conventional media or agent that is not incompatible with the active ingredients of the present disclosure, its use in therapeutic compositions may be used. Supplementary active ingredients also can be incorporated into the compositions, provided they do not inactivate the vectors or cells of the compositions.

In some embodiments, the active compositions of the present disclosure may include classic pharmaceutical preparations. Administration of these compositions according to the present disclosure may be via any common route so long as the target tissue is available via that route, but generally including systemic administration. This includes oral, nasal, or buccal. Alternatively, administration may be by intradermal, subcutaneous, intramuscular, intraperitoneal or intravenous injection, or by direct injection into muscle tissue. Such compositions would normally be administered as pharmaceutically acceptable compositions, as described supra.

The active compounds may also be administered parenterally or intraperitoneally. By way of illustration, solutions of the active compounds as free base or pharmacologically acceptable salts can be prepared in water suitably mixed with a surfactant, such as hydroxypropylcellulose. Dispersions can also be prepared in glycerol, liquid polyethylene glycols, and mixtures thereof and in oils. Under ordinary conditions of storage and use, these preparations generally contain a preservative to prevent the growth of microorganisms.

The pharmaceutical forms suitable for injectable use include, for example, sterile aqueous solutions or dispersions and sterile powders for the extemporaneous preparation of sterile injectable solutions or dispersions. Generally, these preparations are sterile and fluid to the extent that easy injectability exists. Preparations should be stable under the conditions of manufacture and storage and should be preserved against the contaminating action of microorganisms, such as bacteria and fungi. Appropriate solvents or dispersion media may contain, for example, water, ethanol, polyol (for example, glycerol, propylene glycol, and liquid polyethylene glycol, and the like), suitable mixtures thereof, and vegetable oils. The proper fluidity can be maintained, for example, by the use of a coating, such as lecithin, by the maintenance of the required particle size in the case of dispersion and by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars or sodium chloride. Prolonged absorption of the injectable compositions can be brought about by the use in the compositions of agents delaying absorption, for example, aluminum monostearate and gelatin.

Sterile injectable solutions may be prepared by incorporating the active compounds in an appropriate amount into a solvent along with any other ingredients (for example as enumerated above) as desired, followed by filtered sterilization. Generally, dispersions are prepared by incorporating the various sterilized active ingredients into a sterile vehicle which contains the basic dispersion medium and the desired other ingredients, e.g., as enumerated above. In the case of sterile powders for the preparation of sterile injectable solutions, the preferred methods of preparation include vacuum-drying and freeze-drying techniques which yield a powder of the active ingredient(s) plus any additional desired ingredient from a previously sterile-filtered solution thereof.

In some embodiments, the compositions of the present disclosure are formulated in a neutral or salt form. Pharmaceutically acceptable salts include, for example, acid addition salts (formed with the free amino groups of the protein) derived from inorganic acids (e.g., hydrochloric or phosphoric acids, or from organic acids (e.g., acetic, oxalic, tartaric, mandelic, and the like. Salts formed with the free carboxyl groups of the protein can also be derived from inorganic bases (e.g., sodium, potassium, ammonium, calcium, or ferric hydroxides) or from organic bases (e.g., isopropylamine, trimethylamine, histidine, procaine) and the like.

Upon formulation, solutions are preferably administered in a manner compatible with the dosage formulation and in such amount as is therapeutically effective. The formulations may easily be administered in a variety of dosage forms such as injectable solutions, drug release capsules and the like. For parenteral administration in an aqueous solution, for example, the solution generally is suitably buffered and the liquid diluent first rendered isotonic for example with sufficient saline or glucose. Such aqueous solutions may be used, for example, for intravenous, intramuscular, subcutaneous and intraperitoneal administration. Preferably, sterile aqueous media are employed as is known to those of skill in the art, particularly in light of the present disclosure. By way of illustration, a single dose may be dissolved in 1 ml of isotonic NaCl solution and either added to 1000 ml of hypodermoclysis fluid or injected at the proposed site of infusion, (see for example, “Remington's Pharmaceutical Sciences” 15^{th} Edition, pages 1035-1038 and 1570-1580). Some variation in dosage will necessarily occur depending on the condition of the subject being treated. The person responsible for administration will, in any event, determine the appropriate dose for the individual subject. Moreover, for human administration, preparations should meet sterility, pyrogenicity, general safety and purity standards as required by FDA Office of Biologics standards.

In some embodiments, the LEMD2 coding sequence described herein may be delivered to the patient using adoptive cell transfer (ACT). In adoptive cell transfer, one or more expression constructs are provided ex vivo to cells which have originated from the patient (autologous) or from one or more individual(s) other than the patient (allogeneic). The cells are subsequently introduced or reintroduced into the patient. Thus, in some embodiments, one or more nucleic acids encoding LEMD2 are provided to a cell ex vivo before the cell is introduced or reintroduced to a patient.

### V. DEFINITIONS

The terms “polynucleotide,” “nucleic acid” and “transgene” are used interchangeably herein to refer to all forms of nucleic acid, oligonucleotides, including deoxyribonucleic acid (DNA) and ribonucleic acid (RNA) and polymers thereof. Polynucleotides include genomic DNA, cDNA and antisense DNA, and spliced or unspliced mRNA, rRNA, tRNA and inhibitory DNA or RNA (RNAi, e.g., small or short hairpin (sh)RNA, microRNA (miRNA), small or short interfering (si)RNA, trans-splicing RNA, or antisense RNA). Polynucleotides can include naturally occurring, synthetic, and intentionally modified or altered polynucleotides (e.g., variant nucleic acid). Polynucleotides can be single stranded, double stranded, or triplex, linear or circular, and can be of any suitable length. In discussing polynucleotides, a sequence or structure of a particular polynucleotide may be described herein according to the convention of providing the sequence in the 5′ to 3′ direction. A nucleic acid “backbone” can be made up of a variety of linkages, including one or more of sugar-phosphodiester linkages, peptide-nucleic acid bonds (“peptide nucleic acids” or PNA; PCT No. WO 95/32305), phosphorothioate linkages, methylphosphonate linkages, or combinations thereof. Sugar moieties of a nucleic acid can be ribose, deoxyribose, or similar compounds with substitutions, e.g., 2′ methoxy or 2′ halide substitutions. Nitrogenous bases can be conventional bases (A, G, C, T, U), analogs thereof (e.g., modified uridines such as 5-methoxyuridine, pseudouridine, or N1-methylpseudouridine, or others); inosine; derivatives of purines or pyrimidines (e.g., N⁴-methyl deoxyguanosine, deaza- or aza-purines, deaza- or aza-pyrimidines, pyrimidine bases with substituent groups at the 5 or 6 position (e.g., 5-methylcytosine), purine bases with a substituent at the 2, 6, or 8 positions, 2-amino-6-methylaminopurine, O⁶-methylguanine, 4-thio-pyrimidines, 4-amino-pyrimidines, 4-dimethylhydrazine-pyrimidines, and O⁴-alkyl-pyrimidines; U.S. Pat. No. 5,378,825 and PCT No. WO 93/13121). For general discussion see, Adams et al., 1992). Nucleic acids can include one or more “abasic” residues where the backbone includes no nitrogenous base for position(s) of the polymer (U.S. Pat. No. 5,585,481). A nucleic acid can comprise only conventional RNA or DNA sugars, bases and linkages, or can include both conventional components and substitutions (e.g., conventional bases with 2′ methoxy linkages, or polymers containing both conventional bases and one or more base analogs). Nucleic acid includes “locked nucleic acid” (LNA), an analogue containing one or more LNA nucleotide monomers with a bicyclic furanose unit locked in an RNA mimicking sugar conformation, which enhance hybridization affinity toward complementary RNA and DNA sequences (Vester & Wengel, 2004). RNA and DNA have different sugar moieties and can differ by the presence of uracil or analogs thereof in RNA and thymine or analogs thereof in DNA.

A nucleic acid encoding a polypeptide often comprises an open reading frame that encodes the polypeptide. Unless otherwise indicated, a particular nucleic acid sequence also includes degenerate codon substitutions.

Nucleic acids can include one or more expression control or regulatory elements operably linked to the open reading frame, where the one or more regulatory elements are configured to direct the transcription and translation of the polypeptide encoded by the open reading frame in a mammalian cell. Non-limiting examples of expression control/regulatory elements include transcription initiation sequences (e.g., promoters, enhancers, a TATA box, and the like), translation initiation sequences, mRNA stability sequences, poly A sequences, secretory sequences, and the like. Expression control/regulatory elements can be obtained from the genome of any suitable organism.

As used herein, “AAV” refers to an adeno-associated virus vector. As used herein, “AAV” refers to any AAV serotype and variant, including but not limited to an AAV1, AAV2, AAV3, AAV4, AAV5, AAV6, AAV7, AAV8, AAVrh10 (see, e.g., SEQ ID NO: 81 of U.S. Pat. No. 9,790,472, which is incorporated by reference herein in its entirety), AAVrh74 (see, e.g., SEQ ID NO: 1 of US 2015/0111955, which is incorporated by reference herein in its entirety), AAV9 vector, AAV9P vector (also known as AAVMYO, see, Weinmann et al., 2020,), and Myo-AAV vectors described in Tabebordbar et al., 2021, (e.g., MyoAAV 1A, 2A, 3A, 4A, 4C, or 4E), wherein the number following AAV indicates the AAV serotype. The term “AAV” can also refer to any known AAV (vector) system. In some embodiments, the AAV vector is a single-stranded AAV (ssAAV). In some embodiments, the AAV vector is a double-stranded AAV (dsAAV). Any variant of an AAV vector or serotype thereof, such as a self-complementary AAV (scAAV) vector, is encompassed within the general terms AAV vector, AAV1 vector, etc. See, e.g., McCarty et al., 2001, Naso et al., 2017, and references cited therein for detailed discussion of various AAV vectors. Structurally, AAVs are small (25 nm), single-DNA stranded non-enveloped viruses with an icosahedral capsid. Naturally occurring or engineered AAV serotypes and variants that differ in the composition and structure of their capsid protein have varying tropism, i.e., ability to transduce different cell types. When combined with active promoters, this tropism defines the site of gene expression.

A “promoter” refers to a nucleotide sequence, usually upstream (5′) of a coding sequence, which directs and/or controls the expression of the coding sequence by providing the recognition for RNA polymerase and other factors required for proper transcription. “Promoter” includes a minimal promoter that is a short DNA sequence comprised of a TATA-box and optionally other sequences that serve to specify the site of transcription initiation, to which regulatory elements are added for control of expression. A “heterologous promoter” is a promoter that is distinct from a native promoter, i.e., a native being that which is associated in nature with a coding region.

An “enhancer” is a DNA sequence that can stimulate transcription activity and may be an innate element of the promoter or a heterologous element that enhances the level or tissue specificity of expression. It is capable of operating in either orientation (5′->3′ or 3′->5′) and may be capable of functioning even when positioned either upstream or downstream of the promoter.

Promoters and/or enhancers may be derived in their entirety from a native gene or be composed of different elements derived from different elements found in nature, or even be comprised of synthetic DNA segments. A promoter or enhancer may comprise DNA sequences that are involved in the binding of protein factors that modulate/control effectiveness of transcription initiation in response to stimuli, physiological or developmental conditions.

Non-limiting examples include SV40 early promoter, mouse mammary tumor virus LTR promoter; adenovirus major late promoter (Ad MLP); a herpes simplex virus (HSV) promoter, a cytomegalovirus (CMV) promoter such as the CMV immediate early promoter region (CMVIE), a rous sarcoma virus (RSV) promoter, pol II promoters, pol III promoters, synthetic promoters, hybrid promoters, and the like. In addition, sequences derived from non-viral genes, such as the murine metallothionein gene, will also find use herein. Exemplary constitutive promoters include the promoters for the following genes which encode certain constitutive or “housekeeping” functions: hypoxanthine phosphoribosyl transferase (HPRT), dihydrofolate reductase (DHFR), adenosine deaminase, phosphoglycerol kinase (PGK), pyruvate kinase, phosphoglycerol mutase, the actin promoter, and other constitutive promoters known to those of skill in the art. In addition, many viral promoters function constitutively in eukaryotic cells. These include: the early and late promoters of SV40; the long terminal repeats (LTRs) of Moloney Leukemia Virus and other retroviruses; and the thymidine kinase promoter of Herpes Simplex Virus, among many others. Accordingly, any of the above-referenced constitutive promoters can be used to control transcription of a heterologous gene insert.

A “transgene” is used herein to conveniently refer to a nucleic acid sequence/polynucleotide that is intended or has been introduced into a cell or organism. Transgenes include any nucleic acid, such as a gene that encodes an inhibitory RNA or polypeptide or protein and are generally heterologous with respect to naturally occurring AAV genomic sequences.

The term “transduce” refers to introduction of a nucleic acid sequence into a cell or host organism by way of a vector (e.g., a viral particle). Introduction of a transgene into a cell by a viral particle is can therefore be referred to as “transduction” of the cell. The transgene may or may not be integrated into genomic nucleic acid of a transduced cell. If an introduced transgene becomes integrated into the nucleic acid (genomic DNA) of the recipient cell or organism it can be stably maintained in that cell or organism and further passed on to or inherited by progeny cells or organisms of the recipient cell or organism. Finally, the introduced transgene may exist in the recipient cell or host organism extra chromosomally, or only transiently. A “transduced cell” is therefore a cell into which the transgene has been introduced by way of transduction. Thus, a “transduced” cell is a cell into which, or a progeny thereof in which a transgene has been introduced. A transduced cell can be propagated, the transgene transcribed, and the encoded inhibitory RNA or protein expressed. For gene therapy uses and methods, a transduced cell can be in a mammal.

A nucleic acid/transgene is “operably linked” when it is placed into a functional relationship with another nucleic acid sequence. A nucleic acid/transgene encoding and RNAi or a polypeptide, or a nucleic acid directing expression of a polypeptide may include an inducible promoter, or a tissue-specific promoter for controlling transcription of the encoded polypeptide. A nucleic acid operably linked to an expression control element can also be referred to as an expression cassette.

As used herein, the terms “modify” or “variant” and grammatical variations thereof, mean that a nucleic acid, polypeptide or subsequence thereof deviates from a reference sequence. Modified and variant sequences may therefore have substantially the same, greater or less expression, activity or function than a reference sequence, but at least retain partial activity or function of the reference sequence. A particular type of variant is a mutant protein, which refers to a protein encoded by a gene having a mutation, e.g., a missense or nonsense mutation.

A “nucleic acid” or “polynucleotide” variant refers to a modified sequence which has been genetically altered compared to wild-type. The sequence may be genetically modified without altering the encoded protein sequence. Alternatively, the sequence may be genetically modified to encode a variant protein. A nucleic acid or polynucleotide variant can also refer to a combination sequence which has been codon modified to encode a protein that still retains at least partial sequence identity to a reference sequence, such as wild-type protein sequence, and also has been codon-modified to encode a variant protein. For example, some codons of such a nucleic acid variant will be changed without altering the amino acids of a protein encoded thereby, and some codons of the nucleic acid variant will be changed which in turn changes the amino acids of a protein encoded thereby.

The terms “protein” and “polypeptide” are used interchangeably herein. The “polypeptides” encoded by a “nucleic acid” or “polynucleotide” or “transgene” disclosed herein include partial or full-length native sequences, as with naturally occurring wild-type and functional polymorphic proteins, functional subsequences (fragments) thereof, and sequence variants thereof, so long as the polypeptide retains some degree of function or activity. Accordingly, in methods and uses of the disclosure, such polypeptides encoded by nucleic acid sequences are not required to be identical to the endogenous protein that is defective, or whose activity, function, or expression is insufficient, deficient or absent in a treated mammal.

An example of an amino acid modification is a conservative amino acid substitution or a deletion. In particular embodiments, a modified or variant sequence retains at least part of a function or activity of the unmodified sequence (e.g., wild-type sequence).

Another example of an amino acid modification is a targeting peptide introduced into a capsid protein of a viral particle. Peptides have been identified that target recombinant viral vectors or nanoparticles to various organs and tissues.

A “variant” of a molecule is a sequence that is substantially similar to the sequence of the native molecule. For nucleotide sequences, variants include those sequences that, because of the degeneracy of the genetic code, encode the identical amino acid sequence of the native protein. Naturally occurring allelic variants such as these can be identified with the use of molecular biology techniques, as, for example, with polymerase chain reaction (PCR) and hybridization techniques. Variant nucleotide sequences also include synthetically derived nucleotide sequences, such as those generated, for example, by using site-directed mutagenesis, which encode the native protein, as well as those that encode a polypeptide having amino acid substitutions. Generally, nucleotide sequence variants of the disclosure will have at least 40%, 50%, 60%, to 70%, e.g., 71%, 72%, 73%, 74%, 75%, 76%, 77%, 78%, to 79%, generally at least 80%, e.g., 81%-84%, at least 85%, e.g., 86%, 87%, 88%, 89%, 90%, 91%, 92%, 93%, 94%, 95%, 96%, 97%, to 98%, sequence identity to the native (endogenous) nucleotide sequence. In certain embodiments, the variant is biologically functional (i.e., retains 5%, 10%, 15%, 20%, 25%, 30%, 35%, 40%, 45%, 50%, 55%, 60%, 65%, 70%, 75%, 80%, 85%, 90%, 95%, 99% or 100% of activity or function of wild-type).

“Conservative variations” of a particular nucleic acid sequence refers to those nucleic acid sequences that encode identical or essentially identical amino acid sequences. Because of the degeneracy of the genetic code, a large number of functionally identical nucleic acids encode any given polypeptide. For instance, the codons CGT, CGC, CGA, CGG, AGA and AGG all encode the amino acid arginine. Thus, at every position where an arginine is specified by a codon, the codon can be altered to any of the corresponding codons described without altering the encoded protein. Such nucleic acid variations are “silent variations,” which are one species of “conservatively modified variations.” Every nucleic acid sequence described herein that encodes a polypeptide also describes every possible silent variation, except where otherwise noted. One of skill in the art will recognize that each codon in a nucleic acid (except ATG, which is ordinarily the only codon for methionine) can be modified to yield a functionally identical molecule by standard techniques. Accordingly, each “silent variation” of a nucleic acid that encodes a polypeptide is implicit in each described sequence.

The term “substantial identity” of polynucleotide sequences means that a polynucleotide comprises a sequence that has at least 70%, 71%, 72%, 73%, 74%, 75%, 76%, 77%, 78%, or 79%, or at least 80%, 81%, 82%, 83%, 84%, 85%, 86%, 87%, 88%, or 89%, or at least 90%, 91%, 92%, 93%, or 94%, or even at least 95%, 96%, 97%, 98%, or 99% sequence identity, compared to a reference sequence using one of the alignment programs described using standard parameters. One of skill in the art will recognize that these values can be appropriately adjusted to determine corresponding identity of proteins encoded by two nucleotide sequences by taking into account codon degeneracy, amino acid similarity, reading frame positioning, and the like. Substantial identity of amino acid sequences for these purposes normally means sequence identity of at least 70%, at least 80%, 90%, or even at least 95%.

The term “substantial identity” in the context of a polypeptide indicates that a polypeptide comprises a sequence with at least 70%, 71%, 72%, 73%, 74%, 75%, 76%, 77%, 78%, or 79%, or 80%, 81%, 82%, 83%, 84%, 85%, 86%, 87%, 88%, or 89%, or at least 90%, 91%, 92%, 93%, or 94%, or even, 95%, 96%, 97%, 98% or 99%, sequence identity to the reference sequence over a specified comparison window. An indication that two polypeptide sequences are identical is that one polypeptide is immunologically reactive with antibodies raised against the second polypeptide. Thus, a polypeptide is identical to a second polypeptide, for example, where the two peptides differ only by a conservative substitution.

The terms “treat” and “treatment” refer to both therapeutic treatment and prophylactic or preventative measures, wherein the object is to prevent, inhibit, reduce, or decrease an undesired physiological change or disorder, such as the development, progression or worsening of the disorder. For purposes of this disclosure, beneficial or desired clinical results include, but are not limited to, alleviation of symptoms, diminishment of extent of disease, stabilizing a (i.e., not worsening or progressing) symptom or adverse effect of disease, delay or slowing of disease progression, amelioration or palliation of the disease state, and remission (whether partial or total), whether detectable or undetectable. “Treatment” can also mean prolonging survival as compared to expected survival if not receiving treatment. Those in need of treatment include those already with the condition or disorder as well as those predisposed (e.g., as determined by a genetic assay).

As used herein the specification, “a” or “an” may mean one or more. As used herein in the claim(s), when used in conjunction with the word “comprising,” the words “a” or “an” may mean one or more than one.

The use of the term “or” in the claims is used to mean “and/or” unless explicitly indicated to refer to alternatives only or the alternatives are mutually exclusive, although the disclosure supports a definition that refers to only alternatives and “and/or.” As used herein “another” may mean at least a second or more.

Throughout this application, the term “about” is used to indicate that a value includes the inherent variation of error for the device, the inherent variation in the method being employed to determine the value, the variation that exists among the study subjects, or a value that is within 10% of a stated value.

As used herein, “essentially free,” in terms of a specified component, is used herein to mean that none of the specified component has been purposefully formulated into a composition and/or is present only as a contaminant or in trace amounts. The total amount of the specified component resulting from any unintended contamination of a composition is therefore well below 0.05%, preferably below 0.01%. Most preferred is a composition in which no amount of the specified component can be detected with standard analytical methods.

### VI. SEQUENCES

LEMD2 Gene and PrOtein Sequences

Human LEMD2 mRNA:
atggccggcctgtcggacctggaactgcggcgggagctgcaggccctgggcttccagccaggacccatcaccgacaccacccgg
gatgtctaccgcaacaagctgcgccgcctgcggggcgaggcccggctgcgcgacgaggagcggctgcgggaggaggcccggc
cgcggggcgaggagcggttacgggaagaggcccggttacgcgaggatgcgccgctgcgcgcccggcccgccgcggcctctccg
cgggcggagccctggctctcccagccggcctcgggctcggcctacgcgacccctggggcctacggtgatatccggccctccgcgg
cttcctgggtagggagccgcggcctcgcctatcctgcccgcccggcgcaactcaggcgccgcgcctcggtccggggcagctccga
ggaggacgaggacgcccggacgcccgacagggccacgcagggcccgggtctcgcggcccgccgctggtgggcagcgtctccc
gccccggcgcggctgccttcctccctcctcggtcccgacccgcgcccgggcctgcgggcgactcgagcgggccctgctggcgcg
gcgagggcccggcctgaggtggggcgccggctggagcgctggctctctcggcttctgctctgggccagcctagggctactgctcgt
cttcctgggcatcctttgggtgaagatgggcaagccctcagcgccgcaggaggcggaggacaacatgaagttattgccagtggactg
tgagagaaaaacagatgagttctgtcaggccaagcagaaggcagccttgctggagctgctgcatgaactctacaatttcctggccatc
caagctggtaattttgagtgtggaaatccagagaatctaaaaagcaaatgcattcctgttatggaagcccaagaatatatagccaatgtg
accagcagctcctccgccaagtttgaagccgcactgacctggatactgagcagtaacaaggacgtgggcatctggttgaaaggagaa
gaccagtctgaattggtgacgactgtggacaaggtggtctgcctggaatctgcccacccccgcatgggtgttggctgccgcctgagcc
gggccttgctcactgctgtcaccaacgtgctcatcttcttctggtgcttggcttttttgtgggggctcctaattctcctaaaatatcggtggcg
aaagttagaagaggaggaacaagccatgtatgagatggtgaagaagattatagacgtggtccaggaccattacgtggactgggagca
ggacatggagcgctatccatatgtaggcatcctgcacgtgcgcgacagcttgatccctccacagagccggaggcgcatgaagcgtgt
ctgggaccgagctgtggagttcctggcctccaacgaatcccggatccagacggagtcccaccgcgttgcaggagaggacatgctgg
tgtggagatggactaagccctcttccttctctgactcagagcgataa (SEQ ID NO: 52)
Human LEMD2 protein:
MAGLSDLELRRELQALGFQPGPITDTTRDVYRNKLRRLRGEARLRDEERLREEARPR
GEERLREEARLREDAPLRARPAAASPRAEPWLSQPASGSAYATPGAYGDIRPSAASW
VGSRGLAYPARPAQLRRRASVRGSSEEDEDARTPDRATQGPGLAARRWWAASPAP
ARLPSSLLGPDPRPGLRATRAGPAGAARARPEVGRRLERWLSRLLLWASLGLLLVFL
GILWVKMGKPSAPQEAEDNMKLLPVDCERKTDEFCQAKQKAALLELLHELYNFLAI
QAGNFECGNPENLKSKCIPVMEAQEYIANVTSSSSAKFEAALTWILSSNKDVGIWLK
GEDQSELVTTVDKVVCLESAHPRMGVGCRLSRALLTAVTNVLIFFWCLAFLWGLLIL
LKYRWRKLEEEEQAMYEMVKKIIDVVQDHYVDWEQDMERYPYVGILHVRDSLIPP
QSRRRMKRVWDRAVEFLASNESRIQTESHRVAGEDMLVWRWTKPSSFSDSER (SEQ
ID NO: 53)
Mouse LEMD2 mRNA:
atggccggcctgtcggacctggagttgcggcgagagctgcaggccctgggcttccagccaggccccatcaccgacaccacgcgga
acgtctaccgcaacaagctgcgccgcctgcggggcgaggcccggctgcgcgacgacgagcggctgcgggaggacgccgggcc
gcgggaggacgccgggccgcggggccccgagcggcagcgggaggaggcccggctacgcgaggaagcgccgctgcgcgcgc
ggcccgccgccagcgtcctgcgctcggagccctggccgctgtcgccttccccgccggcgcctagcgcggcctccgacgcctcggg
gccgtacggcaacttcggggcctccgcctctccctgggccgcgagccgcggcctctcctacccgccccacgccgggcccgggccg
ctgcgacgccgcgcctcggtccggggcagctcggaggatgacgaggatacacgaacgccggacaggcacgccccgggccggg
gccgccactggtgggccccgccgtcggcctctgcgcggccgcactcggcgctcctcggcgccgacgctcgccccggcctgaagg
gctcgcgcaccggctcggcgggcgccgggcggacccgacccgaggtgggccggtggctggagcgctgcctgtctcggctcctgc
tctgggccagcctggggctgctgctcggcttcttggccatcctgtgggtgaagatgggcaagccctcggcgccgcaggaggcggag
gacaacatgaaattgttgccggtcgactgtgagagaaaaacagatgagttctgccaggccaagcagaaggcggccctgttggagctg
ctgcacgaactgtacaacttcctggccatccaggcaggtaattttgaatgtggcaacccggagaagctgaagagcaagtgcatcccag
tcctggaggcgcaggagtacatagctaatgtgaccagcagtccctcttcgaggtttaaggctgcgctgacctggatcctgagcagcaa
caaggacgtgggcatctggctgaaaggggaagacccatctgagctggcgactacagtggacaaagtggtctgcctggagtccgccc
ggccccggatgggcataggctgccgcctcagccgcgccctgctcaccgctgtcacccacgtgctcatcttcttctggtgcctggccttc
ctgtgggggctgctgatcctcctgaagtaccgctggcggaagctggaggaggaggagcaggccatgtatgagatggtgaagaagat
catagatgtggttcaggaccactatgtggactgggaacaggacatggagcgctacccctacgtgggcatcctccacgtgcgggacag
tctaatccccccacagagccggcggcgcatgaagcgagtatgggaccgtgctgtggaattcctggcttccaatgaatctcggatccag
actgagtcccaccgagtggccggggaggacatgctggtgtggagatggactaagccttcctctttctctgattcagagcgatag
(SEQ ID NO: 54)
Mouse LEMD2 protein:
MAGLSDLELRRELQALGFQPGPITDTTRNVYRNKLRRLRGEARLRDDERLREDAGPR
EDAGPRGPERQREEARLREEAPLRARPAASVLRSEPWPLSPSPPAPSAASDASGPYGN
FGASASPWAASRGLSYPPHAGPGPLRRRASVRGSSEDDEDTRTPDRHAPGRGRHWW
APPSASARPHSALLGADARPGLKGSRTGSAGAGRTRPEVGRWLERCLSRLLLWASL
GLLLGFLAILWVKMGKPSAPQEAEDNMKLLPVDCERKTDEFCQAKQKAALLELLHE
LYNFLAIQAGNFECGNPEKLKSKCIPVLEAQEYIANVTSSPSSRFKAALTWILSSNKD
VGIWLKGEDPSELATTVDKVVCLESARPRMGIGCRLSRALLTAVTHVLIFFWCLAFL
WGLLILLKYRWRKLEEEEQAMYEMVKKIIDVVQDHYVDWEQDMERYPYVGILHVR
DSLIPPQSRRRMKRVWDRAVEFLASNESRIQTESHRVAGEDMLVWRWTKPSSFSDSE
R (SEQ ID NO: 55)

### VII. EXAMPLES

The following examples are included to demonstrate preferred embodiments of the invention. It should be appreciated by those of skill in the art that the techniques disclosed in the examples which follow represent techniques discovered by the inventor to function well in the practice of the invention, and thus can be considered to constitute preferred modes for its practice. However, those of skill in the art should, in light of the present disclosure, appreciate that many changes can be made in the specific embodiments which are disclosed and still obtain a like or similar result without departing from the spirit and scope of the invention.

### Example 1—Materials & Methods

Mouse models. For the generation of the Lemd2 c.T38>G (Lemd2 KI/KI) mouse model, the inventors designed three sgRNAs in the proximity of the Lemd2 mutation and selected the most efficient one after in vitro validation:

(SEQ ID NO: 1)
#Lemd2-sgRNA-2 5′-gcagctctcgccgcaactcc-3′

Additionally, the inventors designed a donor template consisting of a single-stranded oligodeoxynucleotide (ssODN, IDT Ultramer DNA oligos) including the pathogenic Lemd2 mutation (c.T38>G) and a silent mutation (c.G24>A) to prevent recutting after editing, surrounded by two homology arms (91 nt in the 5′ arm and 36 nt in the 3′ arm):

#Lemd2-ssODN-2
(SEQ ID NO: 2)
5′-ggctgccggcgggagcagttccgggtgcggtgcgcgccggggggggc
gagggggcggtgtcctggccatggccggcctgtcggacctggaattgcgg
cgagagcggcaggccctgggcttccagccaggccccatcaccga-3′

Cas9 mRNA, Lemd2 sgRNA and ssODN were injected into the pronucleus of mouse zygotes. For zygote production, B6C3F1 (6 week-old) female mice were treated for superovulation and mated to B6C3F1 stud males. Zygotes were isolated and transferred to M16 (Brinster's medium for ovum culture with 100 units/mL penicillin and 50 mg/mL streptomycin). Subsequently, zygotes were injected in M2 medium (M16 medium and 20 mM Hepes) and cultured in M16 medium for 1 h at 37° C. Injected zygotes were transferred into the oviducts of pseudo-pregnant ICR female mice.

Tail genomic DNA was extracted from F0 mice and the correct insertion of the mutations was confirmed by Sanger sequencing. F0 mosaics were mated to C57BL6N mice to generate mice heterozygous for the c.T38>G mutation. By intercrossing the heterozygous mice, they generated Lemd2 KI/KI animals. For genotyping, the inventors used Custom TaqMan™ SNP Genotyping Assay (Thermo Fisher, 4332077).

For the Lemd2 cardiac-specific knock-out (cKO) mouse model, the inventors designed three sgRNAs 5′ and three sgRNAs 3′ of exon 1 and selected the most efficient one on each side after in vitro validation.

(SEQ ID NO: 3)
#Lemd2-sgRNA-51 5′-ctcgacgcccatccggagac-3′
(SEQ ID NO: 4)
#Lemd2-sgRNA-33 5′-ccttcggggaatgcctgccg-3′

Additionally, the inventors designed two ssODNs (IDT Ultramer DNA oligos) donor templates consisting of LoxP sites, surrounded by two homology arms (91 nt in the 5′ arm and 36 nt in the 3′ arm).

#Lemd2-ssODN-51
(SEQ ID NO: 5)
5′-ctaacgcagcgttagcacgtggtaaacgttcaatggaatgttgattt
acttaatggatgagctcaatggtgtaagaaaaccaccgccattagctagc
ataacttcgtataatgtatgctatacgaagttatatagagatccggccca
cttgccagtctccggatggg-3′
#Lemd2-ssODN-33
(SEQ ID NO: 6)
5′-cccacacctgagccacaggcagggtcagactccttttgacaaatgaa
ggcccggtggtgccagttttgctgctacaaagctgagaccacggactagt
ataacttcgtataatgtatgctatacgaagttatcaggcattccccgaag
gcagggaagacaagaggggc-3′

Zygotes were injected as described above.

Tail genomic DNA was extracted from F0 mice and the correct insertion of the LoxP sites was confirmed by PCR using the following primers:

(SEQ ID NO: 7)
#Lemd2-5′-Fw 5′-tgttgttacgcccagagtctt-3′
(SEQ ID NO: 8)
#Lemd2-5′-Rv 5′- ctatgtccgccatgatgaaa-3′
(SEQ ID NO: 9)
#Lemd2-3′-Fw 5′- aaagccacacgcacactctt-3′
(SEQ ID NO: 10)
#Lemd2-3′-Rv 5′- gtgccagttttgctgctaca-3′

F0 mosaic mice were mated to C57BL6N mice to generate mice heterozygous for the LoxP sites. By intercrossing the heterozygous mice, the inventors generated Lemd2-floxed animals. By breeding these animals with transgenic mice expressing Myh6-Cre (Jackson laboratory, 011038), the inventors generated Lemd2 cKO mice. For genotyping, the inventors used the abovementioned primers. To validate the Lemd2 exon 1 excision (take out), #Lemd2-5′-Fw and #Lemd2-3′-Rv primers were used.

Histology, immunofluorescence and electron microscopy. All histology was performed by the Research Histo Pathology Core at University of Texas Southwestern. Hearts for routine histology were harvested from euthanized mice while still beating and allowed to pump their chambers free of blood in PBS. Subsequently, hearts were fixed overnight in 4% paraformaldehyde (PFA), then dehydrated, cleared, and paraffin embedded by standard histologic procedures (Shehan & Hrapchak, 1980; Woods & Ellis, 1996). Resulting dorsoventral, 4-chamber embeds were serially sectioned on Leica RM2345 rotary microtomes at 5 μm thickness, for routine hematoxylin and eosin stain (H&E) and Masson's Trichrome collagen staining. Skeletal muscle tissues were flash-frozen in a cryoprotective 3:1 mixture of Tissue Freezing Media (TFM) (Fisher Scientific, 15-183-13) and gum tragacanth (Sigma, G1128) and sectioned on a cryostat. Finally, routine H&E was performed. Images were taken using KEYENCE BZ-X700 series microscope.

For tissue immunofluorescence, heart tissues were fixed overnight at 4° C. with 4% PFA prepared in PBS and cryoprotected with a sucrose gradient: 10% and 20% sucrose for 12 h each at 4° C. Finally, tissues were embedded in TFM (Fisher Scientific, 15-183-13), and sectioned at 10 μm using a Leica CM1950 cryostat. Cryosections were air-dried for 30 min at room temperature, fixed in 4% PFA for 15 min, and washed three times with PBS. Antigen retrieval was then performed on an IHC-Tek™ steamer (IHC World, IW-1102) for 60 min using IHC-Tek™ Epitope Retrieval Solution (IHC World, IW-1100). Subsequently, samples were blocked and permeabilized using 10% goat serum/0.3% Triton X-100 in PBS with mouse on mouse (MOM) blocking solution (Vector Labs, BMK-2202) for 60 min at room temperature. Sections were then incubated overnight at 4° C. with the following primary antibodies: γ-H2AX (CST, 9718S, clone 20E3; 1:100), cTNT (Proteintech, 15513-1-AP; 1:100), cTNT (Thermo Fisher Scientific, MA5-12960, clone 13-11; 1:100), Ki67 (Thermo Fisher Scientific, PA5-19462, 1:200) and HCN4 (Abcam, ab32675, clone SHG 1E5; 1:50) prepared in 5% goat serum/0.3% Tween-20 in PBS. Sections were subsequently washed with 0.01% Triton X-100 in PBS three times and incubated with the corresponding secondary antibodies: Goat anti-rat Alexa 488 (Thermo Fisher Scientific, A-11006, 1:400) and Goat anti-rabbit Alexa 488 (Thermo Fisher Scientific A-11008, 1:400), prepared in 5% goat serum in PBS at room temperature for 1 h. After 60 min of secondary antibody incubation along with DAPI nuclear staining (2 mg/mL), sections were washed with PBS, and mounted in Immu-Mount (Fisher, 9990412) or ProLong Gold antifade reagent with DAPI (Thermo Scientific, P36971) medium. Images were acquired using a Zeiss LSM 800 confocal microscope. TUNEL assay was performed using Click-iT Plus TUNEL Assay for In Situ Apoptosis Detection kit (Thermo Fisher Scientific, C10619) following manufacturer's protocol.

For electron microscopy of the whole heart, mice were perfused with 4% PFA and 1% glutaraldehyde in 0.1 M sodium cacodylate buffer (pH 7.4) and stained with 1% osmium tetroxide. Samples were processed by the University of Texas Southwestern Medical Center Electron Microscopy Core facility. Images were acquired using a JEOL 1400 Plus transmission electron microscope.

Plasmids and cloning. The human open reading frame (ORF) of LEMD2 was purchased in pMGF196 from Addgene (97005). Subsequently, the LEMD2 ORF was subcloned into the retroviral vector pMXs-puro (Cell Biolabs, RTV-012). To obtain the c.T38>G mutation, the inventors used the QuikChange II XL Site-Directed Mutagenesis Kit (Agilent Technologies, 200521).

Cell culture, overexpression and immunofluorescence. Mycoplasma-tested C2C12 (ATCC, CRL-1772) mouse myoblasts and Platinum E cells (Cell Biolabs, RV-101) were cultured in 10% fetal bovine serum with 1% penicillin/streptomycin in Dulbecco's Modified Eagle Medium (DMEM). Platinum E cells were used for retrovirus production. Briefly, cells were transfected with FuGENE6 (Promega, E2692) as per the provider's instructions. Fifteen μg of plasmid were used for 10-cm plate transfection. Forty-eight and seventy-two hours after transfection, supernatants were collected and filtered through a 0.45-μm syringe filter. C2C12 cells were infected twice with viral supernatant supplemented with polybrene (Sigma, H9268) at a final concentration of 8 μg/ml. Forty-eight hours after the first infection, cells were replaced with fresh growth media.

For cell immunofluorescence, C2C12 cells overexpressing pMXs-puro-LEMD2 and pMXs-puro-LEMD2 c.T38>G were differentiated into myotubes for five days in DMEM with 2% horse serum. Subsequently, cells were fixed in 4% PFA for 15 min, washed three times with PBS, permeabilized with 0.3% Triton X-100 for 20 min and blocked with 5% bovine serum albumin (BSA) for 30 min. The LEMD2 antibody (Sigma, HPA017340; 1:500) was used in blocking solution for 2 h at room temperature. Sections were subsequently washed with PBS and stained with the corresponding secondary antibody goat anti-rabbit Alexa 555 (Thermo Fisher Scientifc, A-27039). After secondary antibody incubation, sections were washed with PBS, incubated with DAPI at room temperature for 10 min, and washed twice with PBS before mounting. Images were obtained using a Zeiss LSM 800 confocal microscope.

Western blot analysis. Flash-frozen hearts were pulverized using a tissue crusher and protein was isolated in RIPA buffer (Sigma, R0278) containing protease and phosphatase inhibitors (Roche, #04693159001 & #04906837001). To break genomic DNA, samples were sonicated using Bioruptor Pico (Diagenode) for 10 cycles of 30 s sonication on and 30 s sonication off. Isolated cardiomyocytes were resuspended in RIPA buffer (Sigma, R0278) containing protease and phosphatase inhibitors (Roche, #04693159001 & #04906837001). Subsequently, samples were centrifuged at 12,000 g for 20 min at 4° C. to pellet cell debris. Protein concentration was determined by BCA assay (ThermoFisher, 23225), and equal amounts of protein among samples were used for regular western blot and transferred in polyvinylidene fluoride membrane (Millipore, IPVH00010).

Blocking and antibody incubation were performed in 5% milk or 5% BSA in TBS-Tween 0.1%. The following primary antibodies were used: LEMD2 (Sigma, HPA017340, 1:500) and GAPDH (Sigma, MAB374, clone 6C5; 1:500). Horseradish peroxidase (HRP) conjugated secondary antibodies were used (Bio-Rad, #1706515 and #1706516, 1:5000). Immunodetection was performed using Western Blotting Luminol Reagent (Santa Cruz Biotechnology, sc2048) on a SRX-101A film processor (Konica Minolta) and ChemiDoc™ MP Imaging system (Bio-Rad).

Cardiomyocyte (CMs) isolation. Isolation of CMs was performed as previously described (Gan et al., 2021). Briefly, hearts were digested with 2.4 mg/ml collagenase type II in perfusion buffer (120 mM NaCl, 14.7 mM KCl, 0.6 mM KH₂PO₄, 0.6 mM Na₂HPO₄, 1.2 mM MgSO₄, 10 mM Na-HEPES pH=7.0, 4.6 mM NaHCO₃, 30 mM taurine, 10 mM BDM, 5.5 mM glucose) via Langendorff retroaortic perfusion. After digestion, atria and valves were removed and ventricular tissue alone was gently triturated in stop buffer (perfusion buffer with 10% fetal bovine serum with 12.5 μM CaCl₂)) then filtered through a 250-μm nylon mesh. For evaluation of CM size and morphology, CMs were fixed in 2% PFA for 15 min by adding an equal volume of 4% PFA, centrifuged at 300×g, permeabilized with 0.3% Triton X-100 for 20 min and blocked with 5% BSA for 30 min. After centrifugation and resuspension in 5% BSA, CMs were stained with anti-ACNT2 (Sigma-Aldrich, A7811, clone EA-53; 1:500) and goat anti-mouse Alexa 488 (A-21121) using standard procedures. Cells were coverslipped with ProLong Gold antifade reagent with DAPI (Thermo Scientific, P36971). The area, length and width of CMs were analyzed with ImageJ. Length was taken at the longest line parallel to the sarcomere axis and width at the longest line perpendicular to the sarcomere axis; area was calculated based on the entire cell outline, and approximately 110 CMs were analyzed per sample.

For neonatal CM isolation, the inventors used the mouse/rat CM isolation kit (Cellutron Life Technologies, NC-6031) following manufacturer's instructions. After isolation, cells were plated on collagen and laminin coated glass-bottom plates and kept in culture at 37° C. and 5% CO₂ for at least 48 h. For immunostaining, CMs were fixed in 4% PFA for 10 min, permeabilized with 0.3% Triton X-100 for 10 min and blocked with 10% goat serum for 30 min. CMs were stained with anti-γ-H2AX (CST, 9718S, clone 20E3; 1:200), cTnT (Thermo Fisher Scientific, MA5-12960, clone 13-11; 1:200), cardiac troponin I (Abcam, ab47003, 1:200), lamin B1 (Santa Cruz, sc-374015, clone B-10; 1:50), goat anti-rabbit Alexa 488 (Thermo Fisher Scientific, A-11008, 1:400) and goat anti-mouse IgG1 Alexa 555 (Thermo Fisher Scientific, A-21127, 1:4009) on 3% goat serum using standard procedures. Nuclei were stained with Hoechst 33342 Solution 16.2 mM (Thermo Fisher Scientific, H3570, 1:1500). TUNEL assay was performed using Click-iT Plus TUNEL Assay for In Situ Apoptosis Detection kit (Thermo Fisher Scientific, C10619) following manufacturer's protocol. Images were taken using KEYENCE BZ-X700 series microscope and Zeiss LSM 800 confocal microscope.

Cardiomyocyte confinement experiments. A 6-well static cell confiner device (4D cell) was used to mechanically compress isolated CMs. The device employs micropillars and PDMS pistons to compress cells in the vertical axis generating mechanical stretch (Nader et al., 2021). After isolation, CMs were cultured for at least 48 h and then compressed for 1 hour under a pillar length of 20 μm to induce stretching. After compression, cells were processed for downstream applications.

Cardiomyocyte contractility and calcium transients. The isolation of CMs was performed as previously described (Gan et al., 2021). Briefly, following collagenase perfusion and filtering of the cell suspension through nylon mesh as described above, cells were centrifuged at 100×g for 1 min at room temperature, the supernatant was decanted, cells were resuspended in 10 ml stop buffer (perfusion buffer containing 10% of FBS), and recentrifuged. Cells were successively resuspended in 10 ml stop buffer containing 100 PM, 400 μM, and 900 μM CaCl₂ with 2 min delay before recentrifugation. Cells were resuspended in stop buffer containing 1.2 mM CaCl₂ with 2 μg/ml Fura 2-AM (Thermo Fisher Scientific, F1221) in the dark for 5 min at room temperature, then washed once in stop buffer with 1.2 mM CaCl₂ to remove excess label. The loaded cells were then allowed to settle to the bottom of a chamber with cover slip base, which was mounted on a Motic inverted fluorescence microscope. CM contractility and calcium dynamics measurements were performed using a stepper-switch IonOptix Myocyte Calcium and Contractility System. Cells were electrically paced at 1 Hz with a 5 ms pulse of 20 volts. Sarcomere length and shortening were measured using a Fourier transform of CM Z-line patterns under phase contrast optics using a switching rate of 100 Hz. Fura2 calcium transients were captured simultaneously, using the ratio of Fura2 fluorescence emission at 340/380 nm at a switching rate of 1000 Hz. Offline data measurements were performed using IonWizard 6.0 analysis software. Cells displaying asynchronous contractility, excessive blebbing/dysmorphology, and abnormally high or low shortening fraction or calcium amplitude were ignored for acquisition. No preparation of cells was left for more than 10 min before being replaced with a fresh batch of cells.

Bulk RNA Sequencing. Flash-frozen cardiac samples were homogenized in 1 ml of TRIzol (Thermo Fisher Scientific, 15596026) in Precellys Evolution (3 cycles×20 s at 6800 rpm). RNA as isolated using Rneasy Micro Kit (Qiagen, 74004) as per the provider's instructions. RNA-seq libraries (n=3 mice per genotype) were prepared using KAPA mRNA HyperPrep (Kapa Biosystems, kk8580) kit following manufacturer's specifications. High output 75 cycles single-ended sequencing was performed at the CRI Sequencing Facility at UT Southwestern Medical Center using a NextSeq500 sequencer.

Bioinformatics. RNAseq analyses were conducted in R (v.3.3.2) and Python (v.3.5.4). Trim Galore (world-wide-web at bioinformatics.babraham.ac.uk/projects/trim_galore) was used for quality and adapter trimming. The mouse reference genome sequence and gene annotation data, mm10, were downloaded from Illumina iGenomes (support.illumina.com/sequencing/sequencing_software/igenome.html). The qualities of RNA-sequencing libraries were estimated by mapping the reads onto mouse transcript and ribosomal RNA sequences (Ensembl release 89) using Bowtie (v2.3.4.3) (Langmead & Salzberg, 2012). STAR (v2.7.2b) (Dobin et al., 2013) was employed to align the reads onto the mouse genome, SAMtools (v1.9) (Li et al., 2009) was employed to sort the alignments, and HTSeq Python package (Anders et al., 2015) was employed to count reads per gene. DESeq2 R Bioconductor package (Love et al., 2014) was used to normalize read counts and identify differentially expressed (DE) genes, using FDR-adjusted p-value (Benjamini-Hochberg method) of 0.05 as cutoff. Upstream regulator analysis was based on a custom script to identify transcription factors that regulate differentially expressed genes. Enrichment analysis of gene sets was performed using the Metascape (metascape.org) with the supply of upregulated or downregulated DEGs and using p<0.01 as cutoff.

Gene set enrichment analysis (GSEA). Gene set enrichment analysis was performed using GSEA software (Subramanian et al., 2005; Mootha et al., 200). For the analysis, the inventors provided the software with DEGs and selected 1000 permutations and MsigDB.v7.4chip as platform. P<0.05 was used as cutoff.

Quantitative real-time PCR analysis. Total RNA was extracted from whole hearts using TRIzol (Thermo Fisher Scientific, 15596026) and Rneasy Mini Kit (Qiagen, 74104), and reverse transcribed using iScript Reverse Transcription Supermix (Bio-Rad) with random primers. The quantitative polymerase chain reactions (qPCR) were assembled using KAPA SYBR Fast qPCR Master Mix (KAPA, KK4605). Assays were performed using a QuantStudio 5 Real-Time PCR machine (Applied Biosystems). Expression values were normalized to 18S or Gapdh mRNA and were represented as fold-change. Oligonucleotide sequences of qPCR primers are listed in Supplemental Table 1.

Transthoracic echocardiography. Cardiac function was evaluated by two-dimensional transthoracic echocardiography on conscious mice using a VisualSonics Vevo2100 imaging system. Images were acquired as 2D and M-mode (left parasternal long and short axes) and measurements were averaged from 3 consecutive heart beats of M-mode tracings. M-mode tracings were used to measure LV internal diameter at end diastole (LVIDd) and end systole (LVIDs). Fractional shortening (FS) was calculated according to the following formula: FS (%)=[(LVIDd−LVIDs)/LVIDd]×100. Ejection fraction (EF) as EF (%)=(LVEDV−LVESV)/LVEDV×100. (LVESV, left ventricular end systolic volume, LVEDV, left ventricular end diastolic volume). All measurements were performed by an experienced operator blinded to the study.

Electrocardiography. Mice were anesthetized with 1.5% isoflurane in 02 via facemask (following induction in a chamber containing 5% isoflurane). Rectal temperature was continuously monitored and maintained within 37° C.+/−0.3° C. using a heat pad and heat lamp. The surface ECG (lead II) was recorded using two tiny alligator clip electrodes, contacting the skin of the mouse at the upper and lower front of the chest. The signal was acquired for about 1 minute using Chart (v4.2.3) software. The electrocardiogram (ECG) is recorded and analyzed using a digital acquisition and analysis system (Power Lab/4SP; world-wide-web at adinstruments.com). Corrected QT (QTc) duration was calculated using the Bazett formula (QTc=QT/√RR). All measurements were performed by an experienced operator blinded to the study.

For the β-adrenergic stimulation experiment, 3 administrations of isoproterenol (ISO) dissolved in 0.9% NaCl were delivered, starting with a dose of 40 mg/kg of body weight followed by two consecutive 80 mg/kg of body weight injections, 5 and 15 minutes after the first dose (Forte et al., 2021; Brooks & Conrad, 2009). Electrocardiogram (ECG) was recorded for 35 minutes after the first injection.

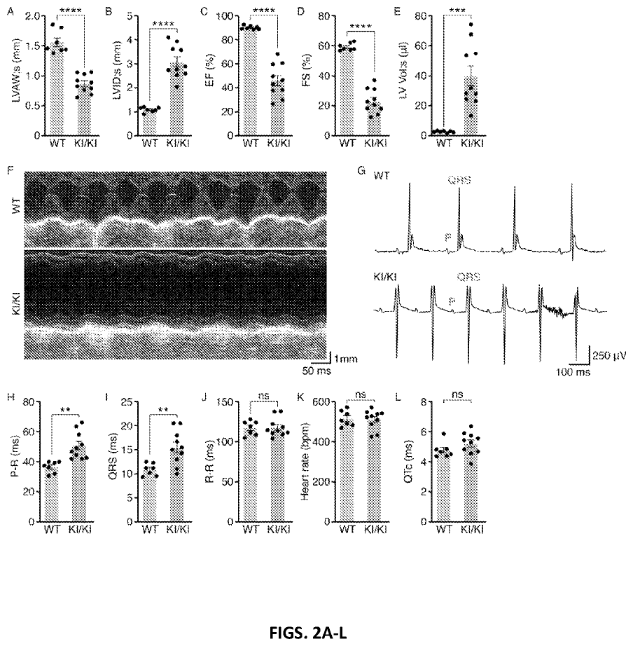

Lemd2 gene therapy. The mouse LEMD2 ORF was purchased from Addgene (plasmid #120245) and cloned into the previously generated AAV serotype 9 vector under the control of the cTnT promoter (Makarewich et al., 2020). AAVs were prepared by the Boston Children's Hospital Viral Core, as previously described (Brinkman et al., 2014). Intraperitoneal injection of P4 Lemd2 KI/KI mice was performed using an ultrafine needle (31 gauge) with 80 μl of saline solution containing the AAV9-Lemd2 viruses (5×10¹³ vg per kg). The AAV9-Lemd2 treatment was unblinded for mouse genotypes and data were compared to untreated WT and Lemd2 KI/KI groups shown in FIGS. 2A-F that were not assessed contemporaneously.

Data availability. All data presented in this study are available in the main text or the Supplemental material. Additional data related to this paper may be requested from the authors. RNA-Sequencing data generated during this study were deposited in Gene Expression Omnibus (GEO) with the accession GSE194218.

Statistics. Data are presented as mean±SEM. Prism software was used for statistical analysis and data plotting. No data were excluded. For physiological, histological and cellular experiments, statistical analysis was performed using two-tailed unpaired t tests or one-way ANOVA with Holm-Sidik correction for multiple comparisons, as indicated in each figure legend. P<0.05 was considered significant, and statistically significant differences are shown with asterisks (*p<0.05, **p<0.01, ***p<0.001, ****p<0.001). Normal distribution was assumed for all variables. For genome-wide analysis, a -fold change >2 and adjusted p<0.05 were used. Sample sizes are indicated in each figure legend. Fiji (Image J) was utilized for image analysis, calculation of nuclear solidity (area/convex area) and fibrosis quantification (Schindelin et al., 2021).

Study approval. All animal procedures were approved by the Institutional Animal Care and Use Committee at the University of Texas Southwestern Medical Center.

### Example 2—Results

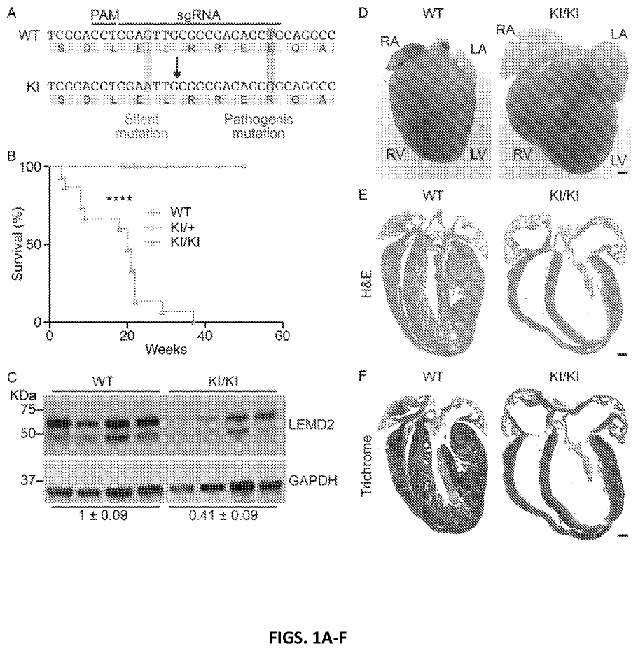

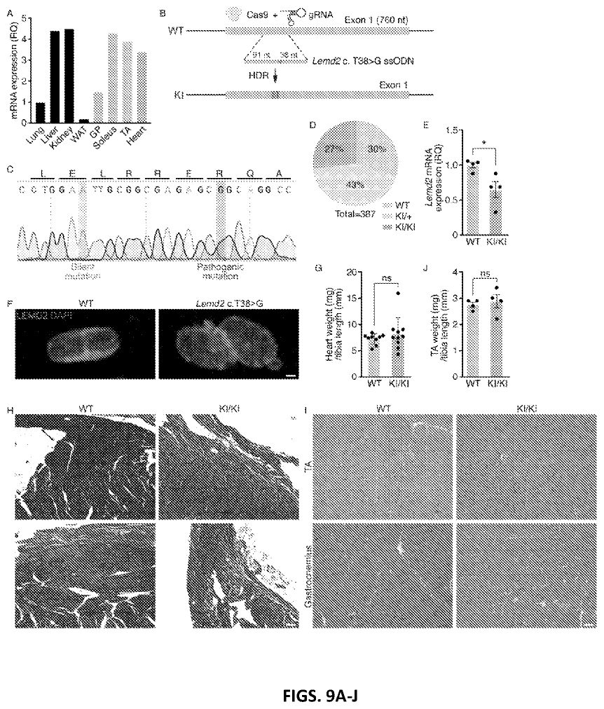

Generation and analysis of Lemd2 KI/KI mice. Lemd2 mRNA is ubiquitously expressed across adult mouse tissues, with relatively higher expression in heart, skeletal muscle, liver and kidney compared to lung and white adipose tissue (FIG. 9A). To investigate the molecular mechanisms underlying cardiomyopathy caused by the Lemd2 c.T38>G mutation in humans (Abdelfatah et al., 2019), the inventors generated mice carrying the same mutation, using CRISPR-Cas9 technology (FIG. 9B). Using a single-stranded oligonucleotide (ssODN) template, they changed nucleotide 38 within codon 13 of the coding region from thymine (T) to guanine (G) (c.T38>G), yielding a leucine to arginine substitution (FIG. 1A). The mutation was confirmed by Sanger sequencing (FIG. 9C).

Homozygous Lemd2 c.T38>G knockin (KI) mice, hereafter referred as KI/KI, were born at expected Mendelian ratios from heterozygous crosses (FIG. 9D). However, while WT and heterozygous (KI/+) mice had normal longevity, the inventors found that KI/KI mice died prematurely with a median lifespan of 20 weeks (FIG. 1B). In this regard, patients carrying the same mutation suffer from sudden death at relatively young ages (between 30-50 years) (Abdelfatah et al., 2019). To determine whether the c.T38>G mutation affects Lemd2 expression, they examined Lemd2 expression at both RNA and protein levels in hearts of Lemd2 KI/KI mice. Quantitative reverse transcription PCR (RT-qPCR) showed that Lemd2 mRNA was modestly but significantly reduced in KI/KI mice compared with WT littermates (FIG. 9E). At the protein level, LEMD2 has two isoforms, expected to be at 57 and 30.7 Kda, respectively. Western blot (WB) analysis of hearts of WT animals revealed that both isoforms displayed a higher size probably because of post-translational modifications. Importantly, both isoforms were reduced (FIG. 1C). To determine the LEMD2 cellular localization, the inventors overexpressed both WT and mutant forms of human LEMD2 using retroviral vectors in C2C12 myoblasts and differentiated them into myotubes. Confocal microscopy showed that both WT and mutant proteins closely associated with the nuclear periphery in myotubes (FIG. 9F). These results showed that LEMD2 protein with the c.T38>G (p.L13>R) mutation localizes similarly as WT LEMD2 protein. Interestingly, this mutant LEMD2 protein also showed normal localization in patient cardiac samples, although protein levels were not altered compared to controls (Abdelfatah et al., 2019).

Hearts from 3-month-old Lemd2 KI/KI mice revealed severe dilation of the atrial and ventricular chambers (FIG. 1D). However, heart weight normalized to tibia length was similar in WT and KI/KI mice (FIG. 9G). Hematoxylin and eosin (H&E) analysis revealed severe DCM in the KI/KI mice, characterized by cardiac chamber dilation and reduced ventricular wall thickness (FIG. 1E). These pathological features indicate that the Lemd2 c.T38>G mutation triggers severe cardiomyopathy in mice. Masson's trichrome staining also showed cardiac fibrosis in the KI/KI mice (FIG. 1F and FIG. 9H). In this regard, a pattern of cardiac fibrosis was also detected in human patients carrying the same LEMD2 mutation (Abdelfatah et al., 2019). The inventors also performed histological examination of different skeletal muscle groups and observed no abnormalities in Lemd2 KI/KI mice (FIG. 9I). Skeletal muscle weight normalized to tibia length was similar in mice of both genotypes (FIG. 9J).

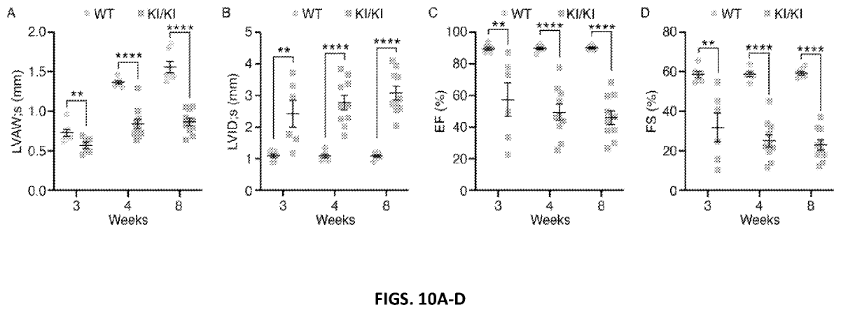

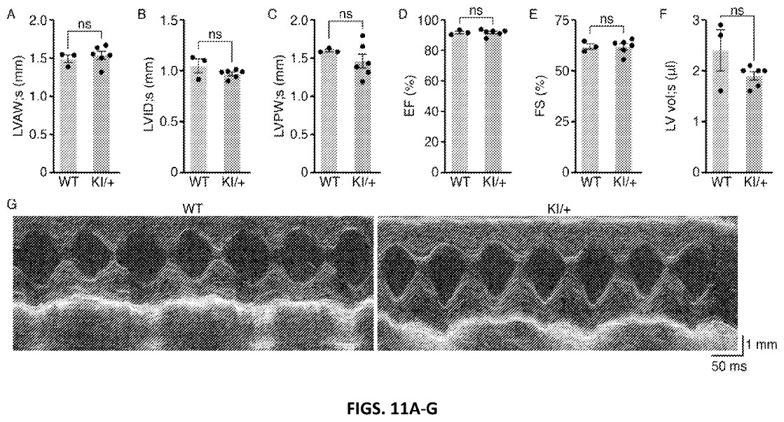

Lemd2 KI/KI mice develop systolic dysfunction, DCM and conduction system abnormalities. The Lemd2 c.T38>G mouse model recapitulates many important pathological phenotypes found in patients. Echocardiography also revealed that the KI/KI hearts showed a significant decrease in the systolic left ventricular anterior wall (LVAW's) thickness (FIG. 2A) and a three-fold increase in the systolic left ventricular internal diameter (LVID's) (FIG. 2B). The ejection fraction (EF) of Lemd2 KI/KI mice was half that of WT mice (FIG. 2C), and fractional shortening (FS) was also dramatically reduced (FIG. 2D). Remarkably, the systolic left ventricular (LV) volume of KI/KI mice was on average forty times greater than that of WT animals (FIG. 2E), presumably as a result of impaired contractility (FIG. 2F). Importantly, Lemd2 KI/KI mice developed these cardiac phenotypes early in life, since the main structural and functional cardiac parameters were significantly altered by three weeks after birth (FIGS. 10A-D). Conversely, the KI/+ mice were overtly normal and displayed completely preserved cardiac function (FIGS. 11A-G). These results indicate that the Lemd2 c.T38>G mutation triggers pathological consequences specifically in the heart only when homozygous.

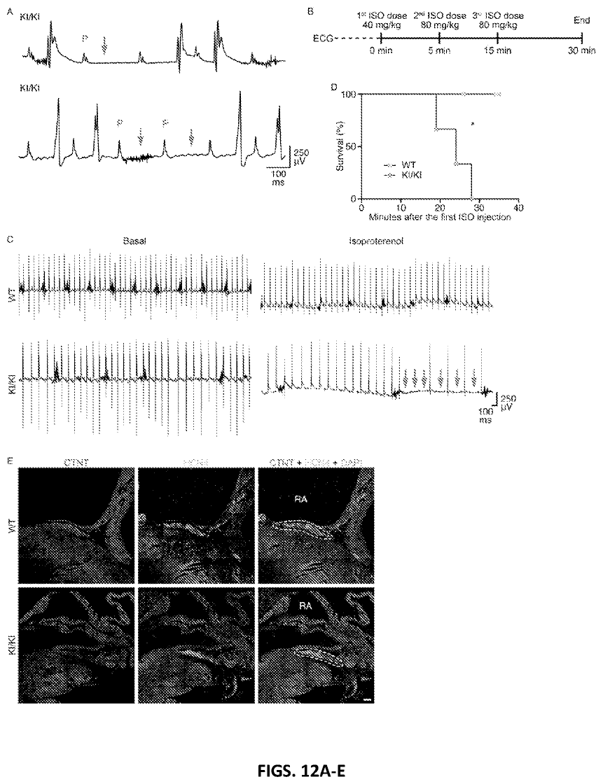

Electrocardiography (ECG) revealed significant cardiac electrical alterations in KI/KI mice, characterized by an increased P-R interval, a hallmark of type I atrioventricular (AV) block (FIGS. 2G-H). These results suggest a delay in the conduction of the electrical signal from the atria to the ventricles through the AV node. The duration of the QRS complex, which corresponds to ventricle depolarization, was also augmented in KI/KI animals (FIG. 2I). Although type I AV block is not a life-threatening condition, the inventors found that these abnormalities worsened to a type II AV block in a subset of KI/KI mice, in which P waves were not followed by a QRS complex, (FIG. 12A). They did not detect alterations in cardiac rhythm or in the corrected QT (QTc) duration; an indicator of cardiac arrythmias; in KI/KI mice under basal conditions (FIGS. 2J-L). To further determine the cause of premature death of the Lemd2 KI/KI mice, the inventors subjected KI/KI mice to acute β-adrenergic stimulation using isoproterenol (ISO) and recorded the ECG for 35 minutes (FIG. 12B). Lemd2 KI/KI mice died before the end of the experiment after showing severe AV blocks, suggesting that cardiac electrical alterations play an important role in the reduced longevity of the Lemd2 KI/KI mice (FIGS. 12 C-D). Finally, to rule out any intrinsic alterations in the AV node, the inventors performed immunostaining for the potassium channel HCN4, a specific marker of the cardiac conduction system (CCS), and corroborated that the morphology and the size of the AV node were similar in WT and KI/KI mice, suggesting that the electrical alterations were not primarily caused by defects in the CCS (FIG. 12E). They speculate that fibrosis accumulation in the heart could account for these alterations in cardiac conduction. Overall, these observations suggest that Lemd2 KI/KI mice die prematurely due to cardiac abnormalities. Moreover, these findings confirmed the histological analysis and demonstrated severe DCM and systolic dysfunction in Lemd2 K/KI mice, resembling the pathological features of patients carrying the same mutation, who also display a reduction in EF and electrical abnormalities (Abdelfatah et al., 2019).

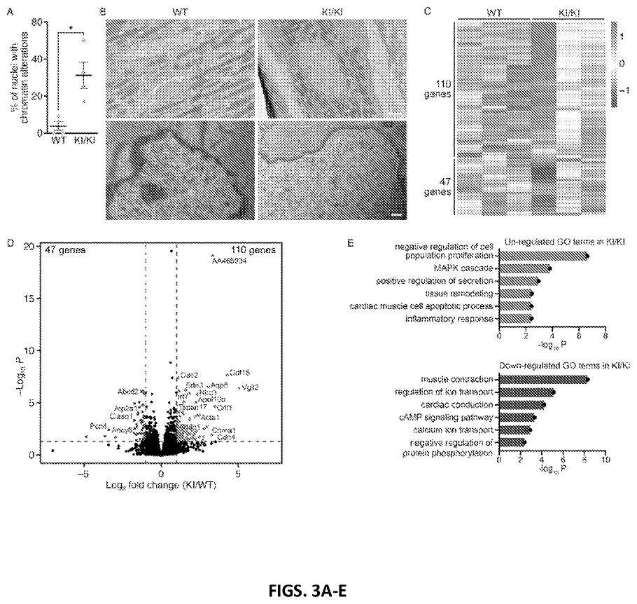

Chromatin abnormalities and transcriptomic alterations in Lemd2 KI/KI mice. To gain mechanistic insights into LEMD2-associated cardiomyopathy, the inventors analyzed the global chromatin organization of the heart by electron microscopy. The repositioning of chromatin close to the NE is an important mechanism for controlling gene expression and this process is largely controlled by NEPs, including LEMD2 (Barrales et al., 2016). The electron-dense nuclear envelope-associated heterochromatin in WT hearts was readily apparent. However, this heterochromatin was almost absent in more than 30% of the KI/KI nuclei (FIG. 3A-B). Thus, these findings indicate that LEMD2 plays a role in chromatin organization in the heart that is impaired by the c.T38>G mutation in the LEM domain.

The loss of NE-associated heterochromatin is expected to cause de-repression of silenced genes in mutant mice. To explore this possibility and to further understand the molecular basis of cardiac dysfunction in mutant hearts, the inventors performed RNA-sequencing (RNA-Seq) of hearts from 2-month-old WT and Lemd2 KT/KI mice. This analysis revealed 110 upregulated and 47 downregulated genes in KI/KI mice compared with their WT littermates (FIGS. 3C-D). Gene Ontology (GO) analysis showed activation of pathways that impair cellular proliferation and promote hypertrophy (FIG. 3E). Among these, the mitogen-activated protein kinase (MAPK) pathway, which has been associated with CM hypertrophy and extracellular matrix remodeling in the heart (Streicher et al., 2010), was enriched in KUKI hearts. Accordingly, genes related to tissue remodeling also showed enriched expression in KI/KI hearts. In this regard, it has been reported that Lemd2 global KO mice also show strong activation of the MAPK pathway, including an increase in ERK1/2, JNK and p38c phosphorylation measured in protein extracts from E10.5 embryos (Tapia et al., 2015). On the other hand, the inventors found repression of pathways related to calcium signaling and muscle function, including muscle contraction, as well as repression of genes associated with cardiac conduction, consistent with the alteration of cardiac conduction in KI/KI mice (FIG. 3E). Among the most up-regulated genes was Gdf15, a member of the transforming growth factor (TGF)-β family that is not expressed in the healthy heart but is induced by p53 signaling as a stress response after hypertrophy or DCM (Xu et al., 2006; Wang et al., 2021). Additionally, the hypertrophy-associated Adap1 gene, encoding the GTPase-activating protein ArfGAP with dual PH domain 1, was also upregulated. (Giguere et al., 2018). Conversely, the calmodulin signaling pathway regulator Pcp4a and the adenylyl cyclase Adcy8 that regulate cardiac rhythmicity were down-regulated in the KI/KI animals (FIG. 3D) (Kim et al., 2014; Moen et al., 2019). Taken together, these results highlight the activation of pro-hypertrophic and anti-proliferative pathways in the Lemd2 KI/KI mice that could explain the cardiac abnormalities in the mutant animals.

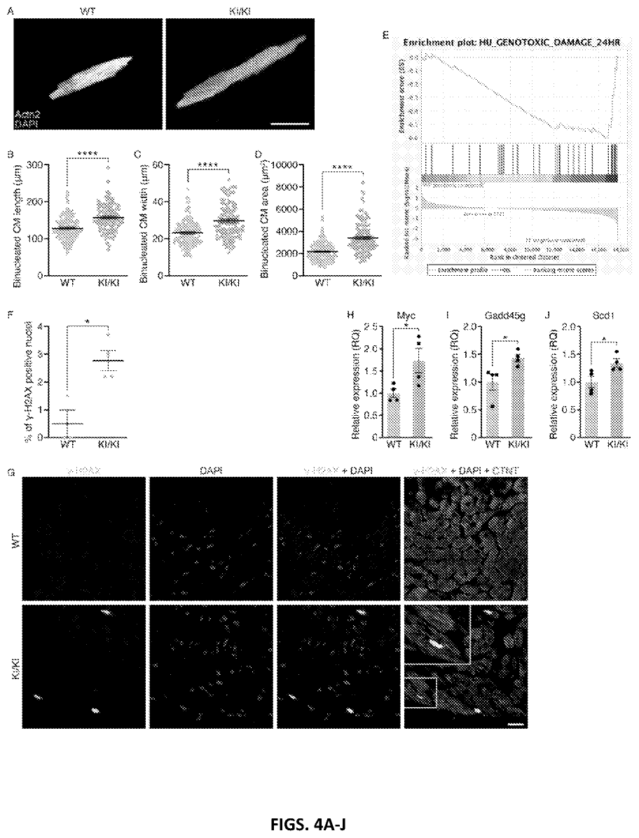

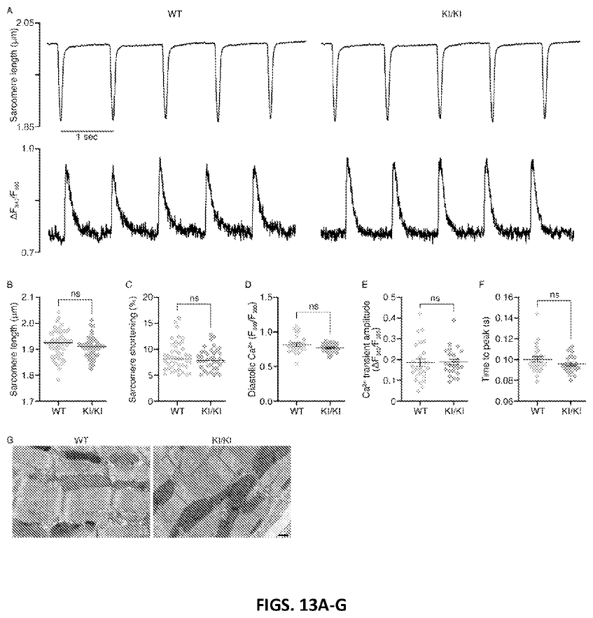

Hypertrophy and DNA damage in Lemd2 KI/KI hearts. To understand the cellular consequences of the transcriptional alterations in Lemd2 KI/KI hearts, the inventors isolated cardiomyocytes (CMs) from 2-month-old WT and Lemd2 KI/KI hearts and performed immunostaining for the sarcomere protein alpha-actinin (ACTN2) to measure CM size and morphology (FIG. 4A). CMs isolated from Lemd2 KI/KI hearts showed a significant increase in length, width and area compared with WT CMs (FIGS. 4B-D). The inventors also subjected isolated CMs to an electrical stimulator (pacing) to study their contractility and calcium handling (FIG. 13A). This assay revealed that the length of sarcomeres as well as their fractional shortening upon electrical stimulation were normal (FIGS. 13B-C). Additionally, using the fluorescent dye Fura-2, the inventors observed that the diastolic calcium levels, the transient amplitude and the time to calcium peak were also preserved in Lemd2 KI/KI CMs (FIGS. 13D-F). Furthermore, transmission electron microscopy (TEM) on cardiac sections revealed normal sarcomere ultrastructure in the Lemd2 KI/KI mice (FIG. 13G).

To further understand the pathogenic mechanisms of the LEMD2-associated cardiomyopathy, the inventors performed Gene Set Enrichment Analysis (GSEA) on cardiac genes differentially-expressed between WT and Lemd2 KI/KI mice. Among the pathways significantly enriched in the KI/KI mice, they found a set of genes related to genotoxic stress, suggesting that DNA damage might play a role in the observed phenotype of the Lemd2 KI/KI mice (FIG. 4E). To confirm this observation, the inventors performed immunofluorescence analysis of the γ-phosphorylation of Ser-139 of histone H2AX, a well-known marker of DNA double-strand break (Collins et al., 2020). They found a greater than 3-fold increase in the number of γ-H2AX positive nuclei in cardiac sections of Lemd2 KI/KI mice compared with WT littermates (FIG. 4F). The number of double-strand break, evidenced by γ-H2AX staining, was readily apparent in Lemd2 KI/KI hearts (FIG. 4G). Additionally, they performed RT-qPCR for genes related to DNA damage. The inventors found that Myc, an activator of the DNA-damage-dependent p53 pathway, and the Gadd45g and Scd1 genes, involved in the DNA damage response, were significantly upregulated in Lemd2 KI/KI hearts (FIGS. 4H-J). These findings suggest the presence of genotoxic stress and DNA damage in the Lemd2 KI/KI hearts.

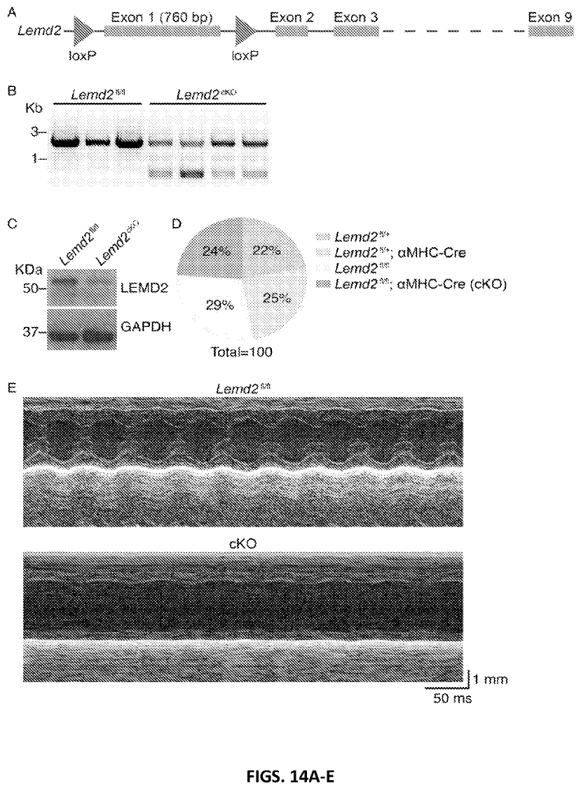

Lemd2 cardiac deficiency leads to cardiomyopathy and premature death in mice. The Lemd2 c.T38>G (Lemd2 KI/KI) mice carry a global hypomorphic mutation, preventing the analysis of cardiac-specific functions of LEMD2. To explore the functions of LEMD2 specifically in CMs, the inventors generated a conditional allele of Lemd2 by introducing two loxP sites flanking the first exon of the Lemd2 gene using CRISPR-Cas9 gene editing, hereafter referred to as Lemd2^{fl/fl}(FIG. 14A). They then bred this mouse line with Myh6-Cre transgenic mice expressing Cre recombinase under the control of the cardiac alpha-myosin heavy chain (ax-MHC) promoter, to enable cardiac-specific deletion of Lemd2 (cKO) (Agah et al., 1997). The inventors confirmed the excision of the floxed alleles (FIG. 14B) and the reduction in both LEMD2 protein isoforms in hearts from Lemd2 cKO animals compared to those from Lemd2^{fl/fl} mice (FIG. 14C). They attribute residual expression of LEMD2 in cardiac extracts to non-CMs, which comprise approximately half of the cells in the heart (Litvinukova et al., 2020).

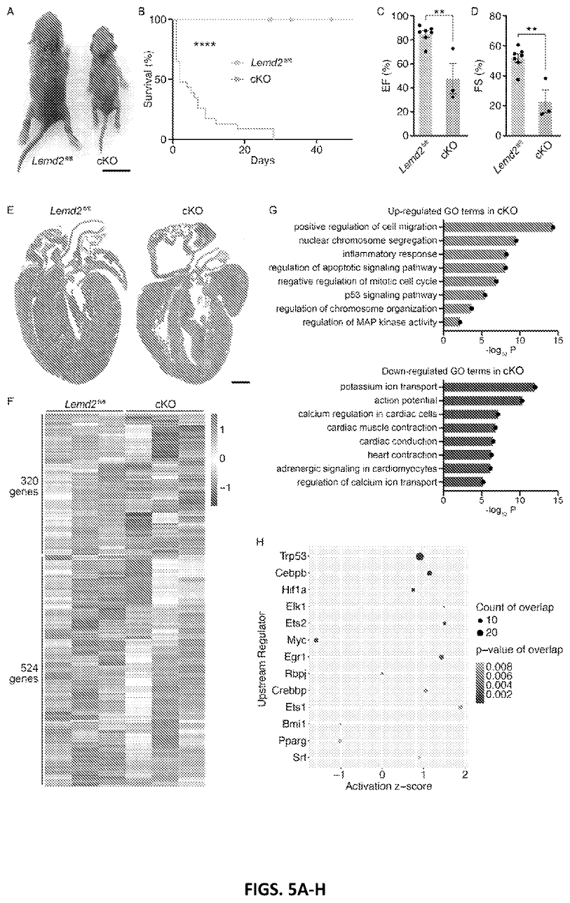

Lemd2 cKO mice were born at Mendelian ratios (FIG. 14D), but developed a striking postnatal phenotype, characterized by a reduction in body size immediately after birth and neonatal lethality, with 50% lethality of cKO mice by two days of age (FIGS. 5A-B). Echocardiographic analysis revealed that cKO mice showed a significant reduction in both ejection fraction and fractional shortening compared with Lemd2^{fl/fl} littermates within the first ten days of life (FIGS. 5C-D). cKO mice displayed systolic dysfunction with severely impaired contraction of the LV (FIG. 14E). Histological analysis showed dilation of atrial and ventricular chambers in cKO mice (FIG. 5E). The finding that Lemd2 cKO mice developed a stronger cardiac phenotype than Lemd2 KI/KI mice suggests that the c.T38>G mutation yields a hypomorphic protein with partially compromised function. The inventors therefore used the cKO animals to study the molecular consequences of LEMD2 loss-of-function in the heart.

The inventors performed transcriptomic analysis by RNA sequencing on cardiac samples from P1 Lemd2^{fl/fl} and cKO mice and identified 844 differentially expressed genes in cKO hearts (FIG. 5F). GO analysis revealed that the most up-regulated pathways were related to apoptosis and negative regulators of proliferation and cell cycle progression (FIG. 5G). Conversely, pathways related to cardiac performance, including cardiac conduction, heart contraction and calcium regulation were down-regulated in cKO hearts. Overall, the transcriptomic dysregulation of the cKO hearts strongly resembled the alterations found in the Lemd2 KI/KI hearts, suggesting that common molecular mechanisms could drive the development of cardiomyopathy in both mouse models.

Interestingly, the inventors also found significant enrichment of pathways related to chromatin organization and activation of the p53 signaling pathway in hearts of cKO mice (FIG. 5G). p53 regulates the expression of many genes related to apoptosis, senescence and the DNA damage response (Mak et al., 2017; Gu et al., 2018). Upstream regulator analysis of the differentially expressed genes identified transcription factors that drive the expression of the genes that were altered in cKO mice. p53 was at the top of the list, controlling the expression of 29 genes, consistent with the activation of the p53 signaling pathway in Lemd2 cKO mice (FIG. 5H). Overall, these results demonstrate that LEMD2 is essential protein for cardiac homeostasis and function and its loss-of-function leads to severe cardiomyopathy in mice with activation of the DNA-damage response and p53-dependent gene expression.

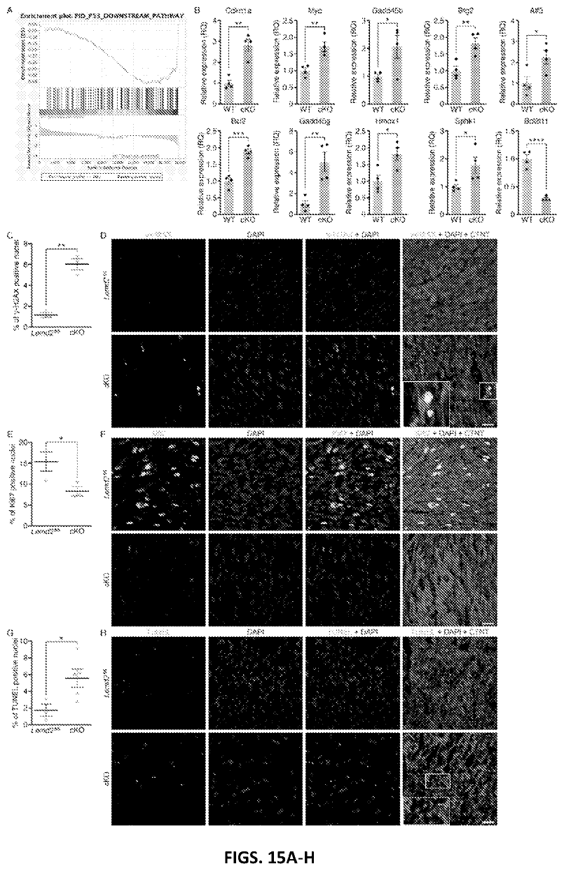

Nuclear envelope deformations, DNA damage and cellular apoptosis in Lemd2 cKO mice. To explore the mechanistic underpinnings of the cardiac abnormalities in Lemd2 cKO mice, the inventors performed GSEA and found that the p53 downstream pathway was highly enriched in cKO hearts (FIG. 15A). They also identified numerous genes belonging to the p53 signaling pathway and involved in the DNA damage response, as being dysregulated in the Lemd2 cKO hearts, as measured by RT-qPCR (FIG. 15B). To validate these results, the inventors performed immunostaining for γ-H2AX on cardiac sections from 5-day-old (P5) Lemd2^{fl/fl} and cKO mice. Histochemical analysis revealed that while Lemd2^{fl/fl} nuclei showed minimal damage, nuclei from cKO hearts were severely affected, showing 6% of the total nuclei positive for H2AX phosphorylation (FIGS. 15C-D). Interestingly, the inventors also found a significant decrease in cellular proliferation, measured by the Ki67 marker (FIGS. 15E-F). Finally, they performed TUNEL staining to detect cellular apoptosis in cKO hearts. This analysis revealed an increase in apoptotic cells in Lemd2 cKO compared to Lemd2^{fl/fl} hearts (FIGS. 15G-H). The reduction in proliferation and the increase in apoptosis could be, at least in part, a direct consequence of high DNA damage. Thus, the percentage of apoptotic nuclei was almost the same as the percentage of cells that showed DNA damage, suggesting that chronic DNA damage triggers cell death in the cKO mice.

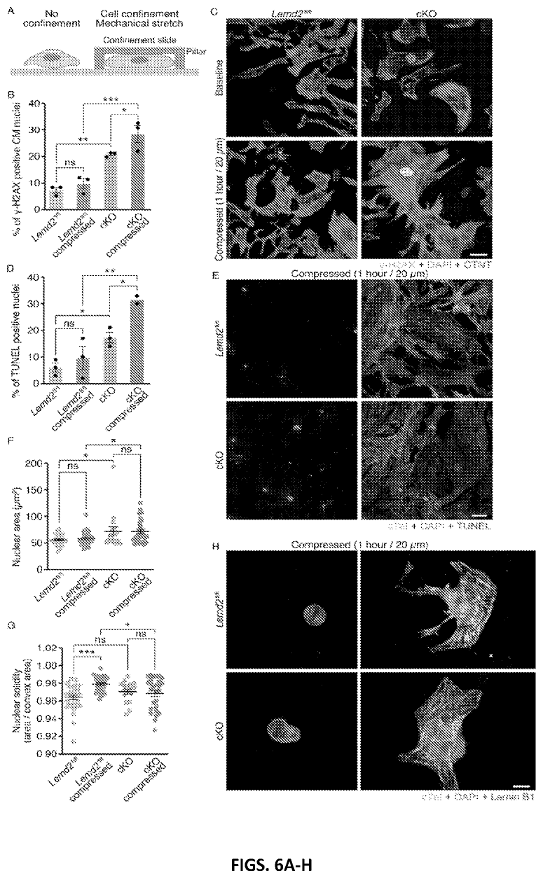

To further validate the causal link between LEMD2 deficiency and DNA damage, the inventors examined if LEMD2 also participates in nuclear envelope stability and mechanotransduction, an important cellular process that senses internal and external mechanical forces and allows cells to respond (Kalukula et al., 2022). They isolated CMs from hearts of Lemd2^{fl/fl} and cKO mice at postnatal day 1 (P1) and subjected them to mechanical stretching using a confiner device (FIG. 6A) (Nader et al., 2021). Importantly, the inventors validated their previous results since under basal conditions cKO CMs exhibited higher levels of DNA damage than those isolated from Lemd2^{fl/fl} hearts (FIGS. 6B-C). Subsequently, when the inventors subjected these cells to 20 m confinement for one hour, they observed that Lemd2^{fl/fl} CMs showed similar DNA damage levels compared to uncompressed cells. Conversely, mechanical stress triggered exacerbated DNA damage in cKO CMs (FIGS. 6B-C). Accordingly, TUNEL staining on the same cells revealed a very similar pattern, strongly suggesting that NE instability leads to cellular apoptosis in Lemd2 cKO hearts (FIGS. 6D-E). These results indicate that, at least in cardiac cells, the LEMD2 protein participates in organizing and stabilizing the chromatin under mechanical stress.

Finally, the inventors investigated the occurrence of nuclear envelope deformations as a potential pathogenic mechanism and source of DNA damage. They stained CMs from hearts of Lemd2^{fl/fl} and cKO P1 mice for the nuclear envelope protein lamin B1 and subjected them to mechanical stress. They noticed that Lemd2-deficient nuclei were bigger than Lemd2^{fl/fl} both at baseline and after compression, which suggests nuclear instability and alterations in chromatin organization (FIG. 6F). To further quantify nuclear deformations, the inventors performed morphometric analysis of the isolated nuclei by calculating their solidity, an indicator of nuclear blebbing. They observed no differences between Lemd2^{fl/fl} and cKO nuclei under basal conditions. However, while control nuclei were able to adapt their morphology to compression by increasing their solidity, Lemd2-deficient CMs failed to adapt to the mechanical stress and showed blebs, suggesting that LEMD2 plays a role in adaptation to mechanical stress (FIGS. 6G-H). Taken together, these findings show that Lemd2-deficiency renders the nuclear envelope more susceptible to deformations under mechanical stress, which in turns generates DNA damage and cellular apoptosis in cKO CMs.

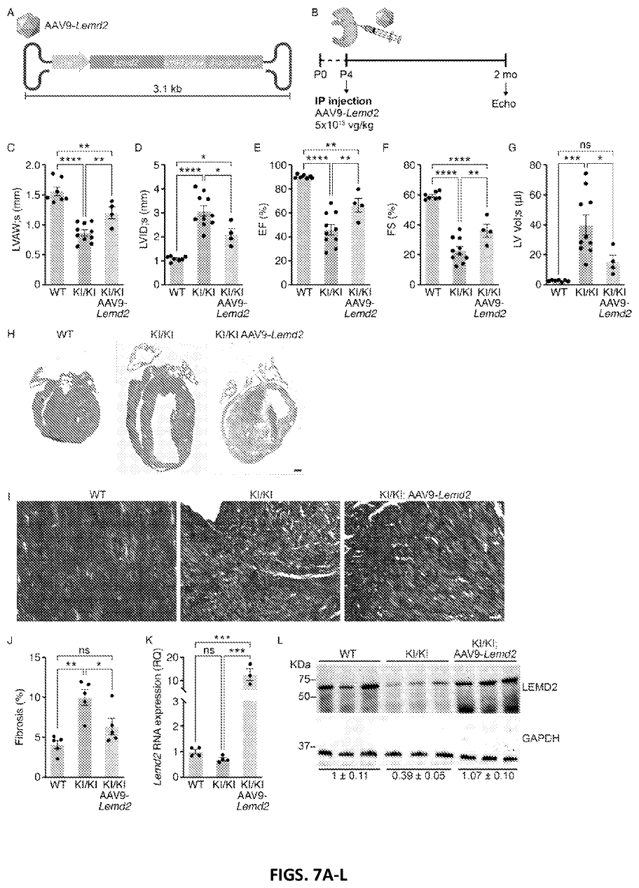

Lemd2 gene therapy improves cardiac function in Lemd2 KI/KI mice. The severity of the LEMD2-associated cardiomyopathy in humans highlights the need for therapeutic approaches aimed at targeting the pathogenic cause of the disease. In this regard, since the c.T38>G mutation causes reduction in LEMD2 mutant protein levels, the inventors hypothesized that an increase in the expression level of the WT full-length LEMD2 protein could provide therapeutic benefits. To test this hypothesis, the inventors engineered adeno-associated virus serotype 9 (AAV9), which displays cardiac tropism, to express the mouse Lemd2 gene under the cardiac-specific promoter of the troponin T (cTnT) gene (FIG. 7A). AAV9 was delivered intraperitoneally (IP) to mice at P4 at a dose of 5×10¹³ vg/kg and echocardiography was performed 2 months later (FIG. 7B) and data were compared with the reference values shown in FIGS. 2A-F. Lemd2 KI/KI mice treated with AAV9-Lemd2 showed an increase in systolic LVAW thickness and a smaller LVID compared with KI/KI untreated mice (FIGS. 7C-D). Moreover, the inventors found an improvement in numerous cardiac functional parameters of the AAV9-treated KI/KI mice, including EF, FS and LV volume, compared with untreated KI/KI animals (FIGS. 7E-G). Histological analysis corroborated that the DCM phenotype and the fibrotic accumulation were substantially ameliorated (FIGS. 7H-J). At the molecular level, Lemd2 mRNA levels were up-regulated more than 10-fold in the hearts of KI/KI mice after AAV treatment compared to WT animals (FIG. 7K). However, protein levels after AAV9-Lemd2 delivery were very similar to those of WT mice, indicating a rescue of the protein expression to physiological levels (FIG. 7L).

SUPPLEMENTAL TABLE 1
Sequences of the primers used for RT-qPCR gene
expression analysis

| Primer ID | Sequence (5′ → 3′) | SEQ ID NO. |
|---|---|---|
| Atf3_Fw | AAGACTGGAGCAAAATGATG | 11 |
| Atf3_Rv | GTCAGGTTAGCAAAATCCTC | 12 |
| Bcl2_Fw | ATGACTGAGTACCTGAACC | 13 |
| Bcl2_Rv | ATATAGTTCCACAAAGGCATC | 14 |
| Bcl2111_Fw | ACGAGTTCAACGAAACTTAC | 15 |
| Bcl2111 Rv | TAGATCCTGTCAATGCCTTC | 16 |
| Btg2_Fw | CTGACCGATCATTACAAACAC | 17 |
| Btg2_Rv | AGACACTTCATAGGGATCAAC | 18 |
| Gadd45g_Fw | CTGCAGATCCATTTCACG | 19 |
| Gadd45g_Rv | TTAGGATTCGAAATGAGGATG | 20 |
| Hmox1_Fw | CATGAAGAACTTTCAGAAGGG | 21 |
| Hmox1 Rv | TAGATATGGTACAAGGAAGCC | 22 |
| Sdc1_Fw | CTTCTGTCATCAAAGAGGTTG | 23 |
| Sdc1_Rv | CAAAGGTGAAGTCTTGTTCTC | 24 |
| Sphk1_Fw | GGTACTCTCATCTCGACTTC | 25 |
| Sphk1_Rv | GCCAGATTTTTAGCTTCCAG | 26 |
| Cdkn1a_Fw | ACCTGATGATACCCAACTAC | 27 |
| Cdkn1a_Rv | CTGTGGCACCTTTTATTCTG | 28 |
| Myc_Fw | TTTTGTCTATTTGGGGACAG | 29 |
| Myc_Rv | CATAGTTCCTGTTGGTGAAG | 30 |
| Gadd45b_Fw | TGCAATCTTCTTTTTACCCC | 31 |
| Gadd45b_Rv | CAAGAGCAAAGTACAAGTCC | 32 |
| Lemd2_Fw | GACTGTGAGAGAAMACAGATG | 33 |
| Lemd2_Rv | CACATTAGCTATGTACTCCTG | 34 |
| Gapdh_Fw | AGGTCGGTGTGAACGGATTTG | 35 |
| Gapdh_Rv | TGTAGACCATGTAGTTGAGGTCA | 36 |
| 18S_Fw | CCATCCAATCGGTAGTAGCG | 37 |
| 18S_Rv | GTAACCCGTTGAACCCCATT | 38 |

### Example 3—Discussion

This is the first study to explore the role of LEMD2, a ubiquitous NEP, in heart disease and cardiac development. These findings reveal the essentiality of LEMD2 for cardiac homeostasis and proper heart function. Mice carrying the same Lemd2 mutation found in humans (c.T38>G) recapitulate the main pathological features of patients with this mutation, including impaired heart function, cardiac fibrosis and premature sudden death (Abdelfatah et al., 2019). In this regard, the Lemd2 c.T38>G mice represent a valuable tool to study LEMD2-associated cardiomyopathy and can be utilized to unravel the molecular mechanisms of this condition as well as providing a preclinical model to test potential therapies. Furthermore, to study LEMD2 function specifically in cardiomyocytes (CMs), the inventors generated the CM-specific Lemd2 conditional knock-out (cKO) mouse model. These animals display a stronger cardiac phenotype than Lemd2 c.T38>G mice, resulting in a median survival of 2 days. These findings further highlight the importance of LEMD2 for normal cardiac function. Additionally, the perinatal lethality of Lemd2 cKO mice suggests that LEMD2 may be also necessary for successful completion of cardiac maturation. Importantly, embryos with a global deletion of Lemd2 die at E11.5, presumably due to cardiac abnormalities (Tapia et al., 2015). In summary, both Lemd2 c.T38>G and cKO mice display a phenotype characterized by systolic dysfunction and DCM.

These data strongly suggest that the Lemd2 c.T38>G mutation is a recessive hypomorphic mutation that causes a reduction in gene expression or protein stability. Thus, the mutant LEMD2 protein is substantially reduced compared to WT animals, which triggers the pathological consequences. This conclusion is reinforced by the fact that the Lemd2 KI/+ heterozygous mice are normal. Moreover, this also explains why the Lemd2 cKO mice, in which LEMD2 is completely absent in CMs, develop more severe disease. The inventors conclude that a threshold level of LEMD2 is required for normal cardiac function and its loss leads to cardiomyopathy.

Alterations in other NEPs such as emerin, another ubiquitously expressed and highly conserved LEM-containing protein, also produce strong phenotypes in both heart and skeletal muscle (Bione et al., 1994). Thus, recessive loss of emerin leads to X-linked EDMD, a severe envelopathy characterized by progressive muscle wasting and cardiomyopathy with conduction defects. (Brull et al., 2018, Brown et al., 2011). Moreover, deficiency in the Lmna gene encoding lamin A/C proteins, or many mutations in the same gene, also cause EDMD-like phenotypes (Cohen et al., 2013; Gao et al., 2020; Auguste et al., 2020; Wada et al., 2019). Conversely, Lemd2 c.T38>G mice do not display pathological defects in skeletal muscle. This could be explained by functional redundancy between LEMD2 and other NEPs or LEM domain-containing proteins. Thus, the inventors speculate that other NEPs may fulfill the function of LEMD2 in muscle and other tissues, as occurs between emerin and LAP1 (Shin et al., 2013), or between emerin and the LEMD2 ortholog in C. elegans (Barkan et al., 2012). In this regard, loss of the muscle-specific NEP NET39 in mice triggers a severe phenotype only in skeletal muscles. Moreover, a direct physical interaction between LEMD2 and NET39 has been reported in C2C12 myotubes (Ramirez-Martinez et al., 2021). In the future, further studies will be necessary to decipher if this interaction is functionally important. If so, this finding may explain why Lemd2 KI/KI mice only develop a phenotype in the heart, where NET39 in not expressed.

Regarding the pathogenic basis of the Lemd2-associated cardiomyopathy, LEMD2 is located in the INM and has been shown to interact with both lamin A and BAF, two important chromatin regulators (Brachner et al., 2005; von Appen et al., 2020). Consistent with such interactions, electron microscopy revealed a dramatic loss of transcriptionally-inactive heterochromatin that is associated with the NE. The control of chromatin organization by NEPs impacts gene expression (Burla et al., 2020). Accordingly, transcriptomic analysis in both Lemd2 mouse models revealed numerous alterations in the expression of genes involved in various molecular pathways. Among them, the inventors found strong activation of the molecular pathway orchestrated by the master regulator p53, which controls a variety of cellular processes, including the DNA damage response and apoptosis (Williams & Schumacher, 2016; Aubrey et al., 2018). Indeed, immunofluorescence analysis on cardiac sections and isolated CMs showed that the double-strand break marker γ-H2AX was present in CM nuclei of the two mutant models. DNA damage could be a direct consequence of nuclear envelope deformations and abnormal mechanotransduction activity in Lemd2-deficient CMs. The inventors hypothesize that these alterations represent a pathogenic mechanism in both Lemd2 models. Damaged CMs also develop hypertrophy and reduced proliferation and undergo cell death. Finally, cardiac fibrosis is triggered as a consequence of the apoptosis. These cellular defects culminate in pathophysiological alterations in Lemd2 mutant mice, including DCM and AV block, which ultimately lead to sudden death. Similar pathogenic mechanisms have been described in other mouse models with mutations in NEPs and lamins (Chen et al., 2019). These findings reinforce the role of NEPs in controlling chromatin organization and suggest that common mechanisms underlie cardiomyopathies associated with different NEPs.

There are currently no available treatments for individuals with cardiomyopathy due to LEMD2 mutations. In this work, the inventors describe an AAV-based gene therapy designed to specifically increase LEMD2 levels in CMs. Using this approach, the inventors were able to improve cardiac morphology and function of Lemd2 KI/KI mice, highlighting the potential of this therapeutic strategy.

It remains to be understood how a change in one amino acid in the LEM domain of LEMD2 impairs protein stability. However, this work is the first to comprehensively study the effects of LEMD2 loss of function in the heart and the pathogenic mechanisms of LEMD2-associated cardiomyopathy. Taken together, these findings indicate that LEMD2 is essential for proper cardiac homeostasis and function through its control of nuclear stability, chromatin organization and gene expression, and its loss of function leads to severe cardiomyopathy driven by DNA damage and p53 activation.

All of the methods disclosed and claimed herein can be made and executed without undue experimentation in light of the present disclosure. While the compositions and methods of this invention have been described in terms of preferred embodiments, it will be apparent to those of skill in the art that variations may be applied to the methods and in the steps or in the sequence of steps of the method described herein without departing from the concept, spirit and scope of the invention. More specifically, it will be apparent that certain agents which are both chemically and physiologically related may be substituted for the agents described herein while the same or similar results would be achieved. All such similar substitutes and modifications apparent to those skilled in the art are deemed to be within the spirit, scope and concept of the invention as defined by the appended claims.

### VIII. REFERENCES

The following references, to the extent that they provide exemplary procedural or other details supplementary to those set forth herein, are specifically incorporated herein by reference.
Abdelfatah et al., JACC Basic Transl Sci. 2019; 4(2):204-21.
Adams et al., The Biochemistry of the Nucleic Acids 5-36, ed., 1 h ed., 1992
Agah et al., J Clin Invest. 1997; 100(1):169-79.
Anders et al., Bioinformatics. 2015; 31(2):166-9.
Aubrey et al., Cell Death Differ. 2018; 25(1):104-13.
Auguste et al., J Clin Invest. 2020; 130(9):4740-58.
Barkan et al., Mol Biol Cell. 2012; 23(4):543-52.
Barrales et al., Genes Dev. 2016; 30(2):133-48.
Bione et al., Nat Genet. 1994; 8(4):323-7.
Boone et al., Mol Genet Genomic Med. 2016; 4(1):77-94.
Brachner et al., J Cell Sci. 2005; 118(Pt 24):5797-810.
Braun & Barrales, Nucleus. 2016; 7(6):523-31.
Brinkman et al., Nucleic Acids Res. 2014; 42(22):e168.
Brooks & Conrad, Comparative Med. 2009; 59(4):339-43.
Brown et al., J Hum Genet. 2011; 56(8):589-94.
Brull et al., Front Physiol. 2018; 9:1533.
Burla et al., Nucleus. 2020; 11(1):205-18.
Chen et al., Circ Res. 2019; 124(6):856-73.
Cheng et al. Nucleus. 2019; 10(1):126-43.
Cohen et al., Hum Mol Genet. 2013; 22(14):2852-69.
Collins et al., Nat Commun. 2020; 11(1):3158.
Dobin et al., Bioinformatics. 2013; 29(1):15-21.
Forte et al., J Cell Mol Med. 2021; 25(1):229-43.
Gan et al., Hum Mol Genet. 2021; 29(21):3504-15.
Gao et al., J Neurosci. 2020; 40(38):7203-15.
Gerbino et al., Front Physiol. 2018; 9:1356.
Giguere et al., Sci Rep. 2018; 8(1):13605.
Gu et al., Cell Death Dis. 2018; 9(2):82.
Huber et al., Mol Cell Biol. 2009; 29(21):5718-28.
Janin et al., Orphanet J Rare Dis. 2017; 12(1):147.
Janin & Gache, Front Physiol. 2018; 9:1277.
Kalukula et al., Nat Rev Mol Cell Biol. 2022; 23:583-602.
Kim et al., J Clin Invest. 2014; 124(11):5027-36.
Langmead & Salzberg, Nat Methods. 2012; 9(4):357-9.
Li et al., Bioinformatics. 2009; 25(16):2078-9.
Litvinukova et al., Nature. 2020; 588(7838):466-72.
Love et al. Genome Biol. 2014; 15(12):550.
Mak et al., Proc Natl Acad Sci USA. 2017; 114(9):2331-6.
Makarewich et al., Circ Res. 2020; 127(10):1340-2.
McCarty et al., Gene Ther. 2001; 8:1248-54
Moen et al., Front Neurosci. 2019; 13:615.
Mootha et al., Nat Genet. 2003; 34(3):267-73.
Nader et al., Cell. 2021; 184(20):5230-46 e22.
Naso et al., BioDrugs 2017; 31:317-334
Pawar & Kutay, Spring Harb Perspect Biol. 2021; 13(9):a040477.
Ramirez-Martinez et al., Nat Commun. 2021; 12(1):690.
Schindelin et al., Nat Methods. 2012; 9(7):676-82.
Schirmer et al., Science. 2003; 301(5638):1380-2.
Shehan & Hrapchak, Battelle Press; 1980.
Shin et al., Dev Cell. 2013; 26(6):591-603.
Shin & Worman, Annu Rev Pathol. 2021.
Streicher et al., Circ Res. 2010; 106(8):1434-43.
Subramanian et al., Proc Natl Acad Sci USA. 2005; 102(43):15545-50.
Tabebordbar et al., 2021, Cell, 184:1-20
Tapia et al., PLoS One. 2015; 10(3):e0116196.
Ulbert et al., FEBS Lett. 2006; 580(27):6435-41.
Ungricht & Kutay, Nat Rev Mol Cell Biol. 2017; 18(4):229-45.
Vester and Wengel, 2004, Biochemistry 43(42):13233-41
Vietri et al., Nat Cell Biol. 2020; 22(7):856-67.
von Appen et al., Nature. 2020; 582(7810):115-8.
Wada et al., PLoS One. 2019; 14(8):e0221512.
Wang et al., Nat Rev Endocrinol. 2021; 17(10):592-607.
Weinmann et al., 2020, Nature Communications, 11:5432
Wilkie et al. Mol Cell Proteomics. 2011; 10(1):M110 003129.
Williams & Schumacher, Cold Spring Harb Perspect Med. 2016; 6(5).
Woods & Ellis, Churchill—Livingstone Press.; 1996.
Xu et al., Circ Res. 2006; 98(3):342-50.

1. An expression construct comprising a coding region for LEM domain-containing protein 2 (LEMD2) under the control of a heterologous promoter.

2. The expression construct of claim 1, wherein said expression construct is a non-viral expression construct.

3. The expression construct of claim 1, wherein said expression construct is a viral expression construct.

4. The expression construct of claim 1, wherein said heterologous protein is a constitutive promoter or an inducible promoter.

5. The expression construct of claim 1, wherein the promoter is a muscle-specific promoter.

6. The expression construct of claim 5, wherein the muscle-specific promoter is a cardiac troponin T (cTnT) promoter.

7. The composition of claim 3, wherein said viral expression construct is a retroviral construct, an adenoviral construct, an adeno-associated viral construct, a poxviral construct, or a herpesviral construct.

8. The expression construct of claim 7, wherein the adeno-associated viral construct comprises a sequence isolated or derived from an AAV vector of serotype 1 (AAV1), 2 (AAV2), 3 (AAV3), 4 (AAV4), 5 (AAV5), 6 (AAV6), 7 (AAV7), 8 (AAV8), 9 (AAV9), 10 (AAV10), 11 (AAV11), MyoAAV, or any combination thereof.

9. The expression construct of claim 7, wherein the AAV vector comprises a sequence isolated or derived from an AAV vector of serotype 9 (AAV9).

10. The expression construct of claim 7, wherein the AAV vector comprises a sequence isolated or derived from an AAV vector of serotype 2 (AAV2).

11. The expression construct of claim 7, wherein the AAV vector comprises a sequence isolated or derived from an AAV2 and a sequence isolated or derived from an AAV9.

12. The expression construct of claim 1, wherein the viral vector is optimized for expression in mammalian cells.

13. The expression construct of claim 3, wherein the vector is optimized for expression in human cells.

14. A composition comprising the expression construct of claim 1.

15. A cell comprising the expression construct of claim 1.

16. The cell of claim 15, wherein the cell is a human cell.

17. The cell of claim 15, wherein the cell is a mouse cell.

18. The cell of claim 16, wherein the human cell is a cardiomyocyte.

19. The cell of claim 15, wherein the cell or human cell is an induced pluripotent stem (iPS) cell.

20. A composition comprising the cell of claim 15.

21. A method of expressing LEM domain-containing protein 2 (LEMD2) in a cell comprising delivering an expression construct of claim 1 to said cell.

22. A method of treating or preventing LEM domain-containing protein 2 (LEMD2) cardiomyopathy in a subject comprising administering to the subject a therapeutically effective amount of a composition comprising the expression construct of claim 1.

23. The method of claim 22, wherein the composition is administered locally.

24. The method of claim 22, wherein the composition is administered directly to cardiac tissue.

25. The method of claim 22, wherein the composition is administered by an infusion or injection.

26. The method of claim 22, wherein the composition is administered systemically.

27. The method of claim 26, wherein the composition is administered by an intravenous infusion or injection.

28. The method of claim 22, wherein administration of said expression construct results one or more of an increase in systolic LVAW thickness as compared with Lemd2 KI/KI untreated mice, a smaller LVID as compared with Lemd2 KI/KI untreated mice, improvement in cardiac functional parameters (e.g., EF, FS and LV volume) as compared with untreated Lemd2 KI/KI animals and/or amelioration of DCM phenotype and fibrotic accumulation compared with Lemd2 KI/KI untreated mice.

29. The method of claim 22, wherein the subject is a neonate, an infant, a child, a young adult, or an adult.

30. The method of claim 22, wherein the subject is male.

31. The method of claim 22, wherein the subject is female.

32. (canceled)

33. A knock-in mouse comprising T38>G mutation in the LEM domain-containing protein 2 (LEMD2).

34. The knock-in mouse of claim 33, wherein said mutation is heterozygous.

35. The knock-in mouse of claim 33, wherein said mutation is homozyogous.

36. A method of making a knock-in mouse strain comprising contacting a murine cell with Cas9, guide RNA (gRNA) targeting LEM domain-containing protein 2 (LEMD2) sequence and a single-stranded oligonucleotide (ssODN) template containing a thymine to guanine (G) substitution in nucleotide 38 (c.T38>G) within codon 13 of the coding region of LEMD2, yielding a leucine to arginine substitution in Lemd2 mouse endogenous gene.
